(19) United States
(12) Patent Application Publication
(10) Pub. No.: US 2025/0333782 A1
(43) Pub. Date: Oct. 30, 2025

(54) COMPOSITIONS INCLUDING AQUEOUS AMINE BORANE COMPLEXES AND POLYNUCLEOTIDES, AND METHODS OF USING THE SAME TO DETECT METHYLCYTOSINE OR HYDROXYMETHYLCYTOSINE

(71) Applicant: ILLUMINA, INC., San Diego, CA (US)

(72) Inventors: Elena Cressina, Cambridge (GB); Mykhailo Vybornyi, Cambridge (GB); Alexandre Hofer, Cambridge (GB); Carole Anastasi, Cambridge (GB)

(21) Appl. No.: 18/852,966

(22) PCT Filed: Mar. 29, 2023
(86) PCT No.: PCT/EP2023/058202

§ 371 Date: Sep. 30, 2024

Related U.S. Application Data
(60) Provisional application No. 63/325,744, filed on Mar. 31, 2022.

(51) Int. Cl. C12Q 1/6869; C12Q 1/26; C12Q 1/6806; C12Q 1/6809

(52) U.S. Cl. CPC C12Q 1/6869; C12Q 1/26; C12Q 1/6806; C12Q 1/6809; C12Y 114/11; C12Q 2600/154

(57) ABSTRACT

Disclosed herein are aqueous compositions that include a pyridine complex and a polynucleotide. The compositions can be used to detect methylcytosine and/or hydroxymethylcytosine in the polynucleotide. In some examples, the compositions may be used as part of a TET-assisted borane sequencing workflow.

### CROSS-REFERENCE TO RELATED APPLICATION

This application is a national stage filing under 35 U.S.C. § 371 of international application number PCT/EP2023/058202 filed Mar. 29, 2023, which claims the benefit of U.S. Provisional Patent Application No. 63/325,744 filed Mar. 31, 2022 and entitled “Compositions Including Aqueous Amine Borane Complexes and Polynucleotides, and Methods of Using the Same to Detect Methylcytosine or Hydroxymethycytosine,” the entire contents of each are incorporated by reference herein.

### FIELD

This application relates to borane complexes, and detecting methylcytosine using borane complexes.

### SEQUENCE LISTING

The instant application contains a Sequence Listing which has been submitted electronically in xml format and is hereby incorporated by reference in its entirety. Said xml copy was created on Oct. 7, 2024, is named 144194_00016_ST26.xml, and is 27,118 bytes in size.

### BACKGROUND

Within living organisms, such as humans, selected cytosines in the genome may become methylated. A common method used to detect methylated cytosines is sodium bisulfite sequencing. One issue with this method is that it often results in greater than 95% of the input DNA being degraded. Borane-containing compounds can be used in various protocols to detect methylated cytosines. However, previously known boranes can also degrade DNA. Thus, new methods and compositions are needed to detect methylated DNA that reduces DNA degradation.

### SUMMARY

Examples provided herein are related to compositions that include a polynucleotide and an aqueous solution in contact with the polynucleotide. In some examples, the aqueous solution includes an amine-borane complex (as such, the amine borane complex may be referred to as an aqueous amine borane complex). Methods of using the compositions to detect methylcytosine or hydroxymethylcytosine in the polynucleotide are described herein.

Some examples herein provide a composition that includes a polynucleotide and an aqueous solution in contact with the polynucleotide. The aqueous solution may include a substituted pyridine borane complex of formula I:

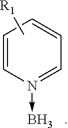

where R₁ includes a heteroatom.

In some examples, the heteroatom is at least one of iodine, nitrogen, bromine, chlorine, fluorine, sulfur, phosphorus, and oxygen.

Some examples herein provide a composition including a polynucleotide and an aqueous solution in contact with the polynucleotide. The aqueous solution may include a substituted pyridine borane complex of formula I:

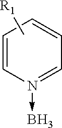

where R₁ includes any one of a sulfur, an oxygen, a nitrogen, a carbonyl group, and a carbon atom.

Some examples herein provide a composition including a polynucleotide and an aqueous solution in contact with the polynucleotide. The aqueous solution may include a substituted pyridine borane complex of formula I:

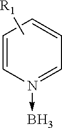

where R₁ provides an enhanced bond strength between the nitrogen and borane (i) as compared to a bond strength of nitrogen and borane in pyridine borane and (ii) as compared to a bond strength of nitrogen and borane in picoline borane.

The following examples are options for any of the above compositions.

In some examples, R₁ is an electron donating group. In some examples, the electron donating group includes an oxygen that is bonded to the pyridine ring. In some examples, the oxygen is part of a hydroxide group or methoxy group. In some examples, the electron donating group includes a nitrogen that is bonded to the pyridine ring. In some examples, the nitrogen is part of an amino group, an amide group, or a carbamate group.

In some examples, R₁ is an electron withdrawing group. In some examples, the electron withdrawing group includes a carbonyl group that is bonded to the pyridine ring. In some examples, the carbonyl group is part of a group including any of an aldehyde, a ketone, a carboxylic acid, an ester, or an amide group. In some examples, the electron withdrawing group includes a carboxylate or a carboxamide.

In some examples, R₁ is ortho to the nitrogen. In some examples, R₁ is meta to the nitrogen. In some examples, R₁ is para to the nitrogen.

In some examples, the pyridine borane complex of formula I has a structure selected from the group consisting of:

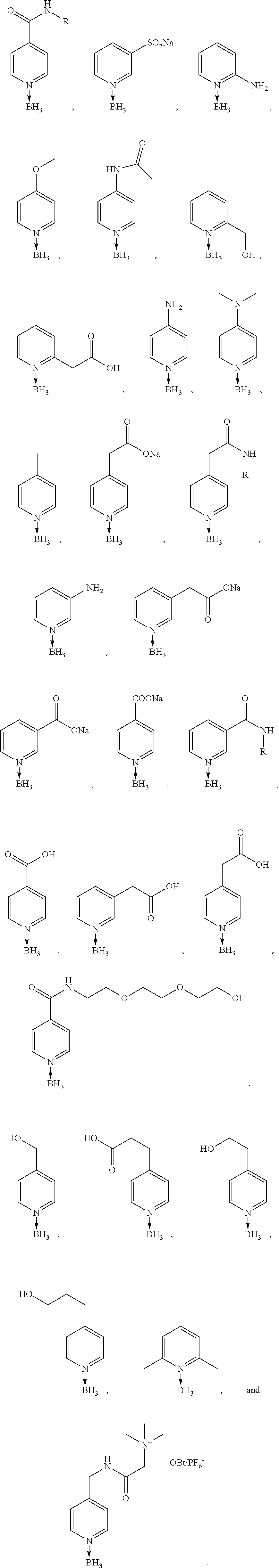

In some examples, the pyridine borane complex is of formula Ia:

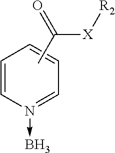

where X is O, NH, or S, and where R₂ includes at least one of O, N, and S.

In some examples, the pyridine borane complex of formula Ia has a structure selected from the group consisting of:

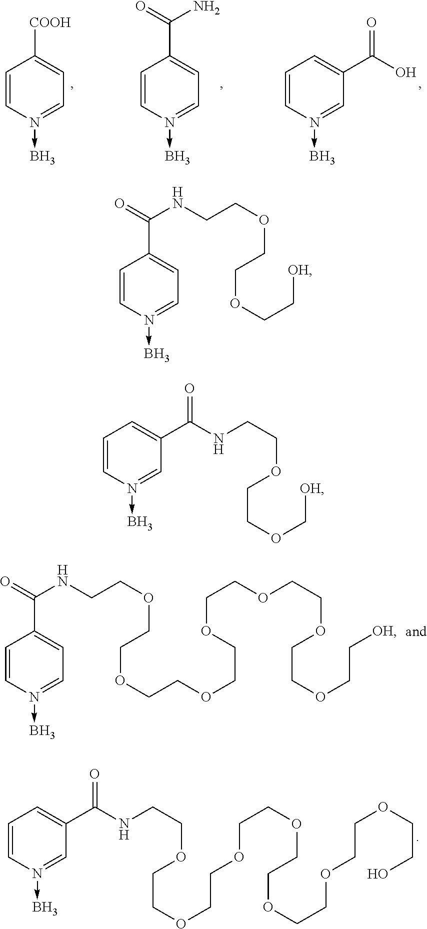

In some examples, the pyridine borane complex is of formula Ib:

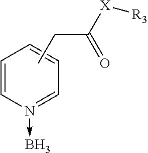

where X is O, NH, or S, and where R₃ includes at least one of O, N, and S.

In some examples, the pyridine borane complex of formula Ib has a structure selected from the group consisting of:

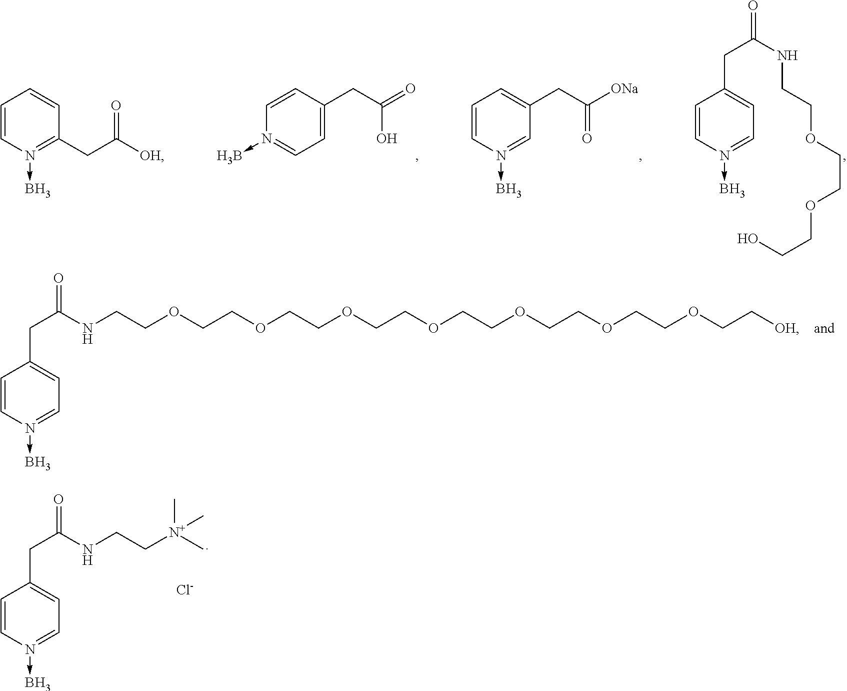

Some examples herein provide a composition including a polynucleotide and an aqueous solution in contact with the polynucleotide. The aqueous solution may include an azole borane complex of formula II

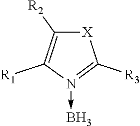

where X is S, O, or NR₄; and
where R₁, R₂, and R₃, independently include at least one of H, C, O, N, and S.

In some examples the azole borane complex of formula II includes a thiazole complex. In some examples, the thiazole complex has a structure selected from the group consisting of:

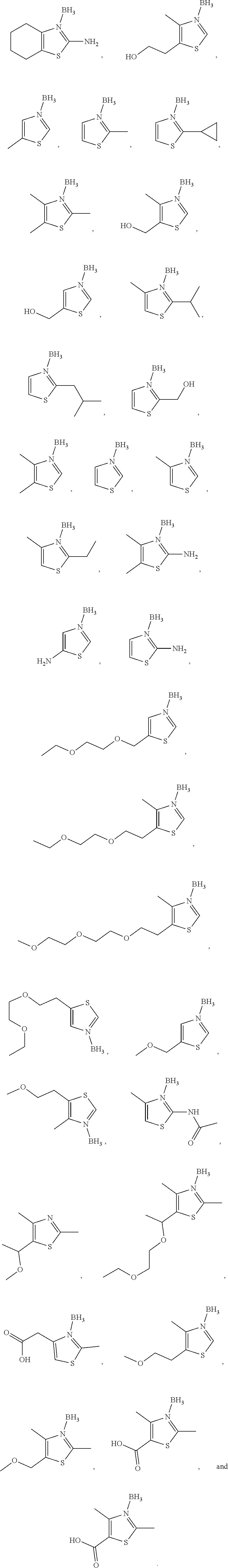

In some examples, the azole borane complex of formula II includes an oxazole complex. In some examples, the oxazole complex has a structure selected from the group consisting of:

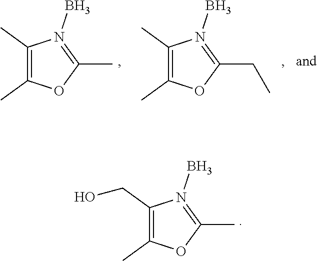

In some examples, the azole borane complex of formula II includes an imidazole complex. In some examples, the imidazole complex has a structure selected from the group consisting of:

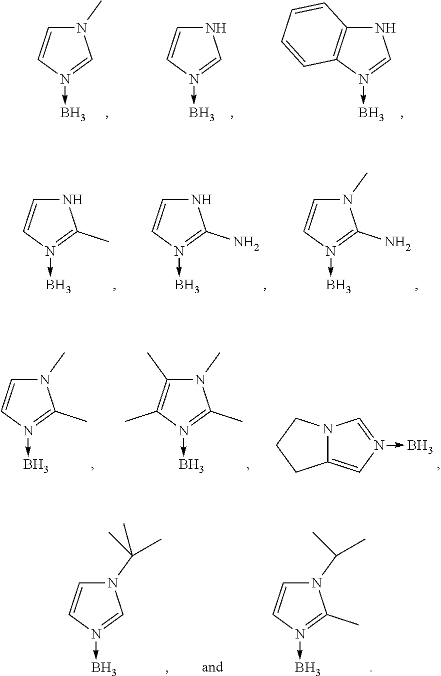

Some examples herein provide a composition including a polynucleotide and an aqueous solution in contact with the polynucleotide. The aqueous solution may include a pyrimidine borane complex of formula III:

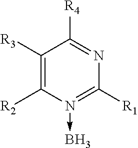

where R₁, R₂, R₃, and R₄ independently include at least one of C, O, N, and S.

In some examples, the pyrimidine borane complex of formula III has a structure selected from the group consisting of:

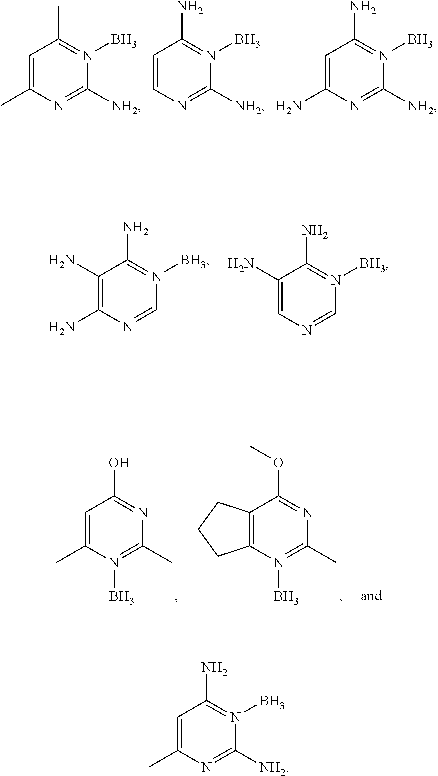

Some examples herein provide a composition including a polynucleotide and an aqueous solution in contact with the polynucleotide. The aqueous solution may include a substituted pyridine borane complex of formula I:

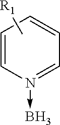

where R₁ includes a cationic moiety.

In some examples, the substituted pyridine borane complex containing a cationic moiety includes any of the following structures:

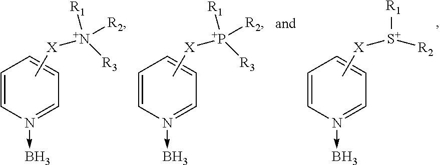

where X is a generic linker group and R₁, R₂, and R₃ include a generic substituent (e.g., an alkyl group) or a hydrogen.

In some examples, the substituted pyridine borane complex containing a cationic moiety includes any of the following structures:

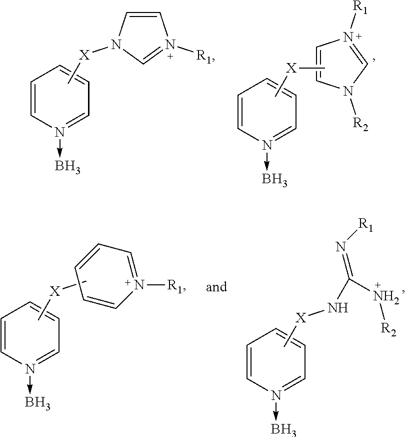

where X is a generic linker group and R₁ and R₂ include a generic substituent (e.g., an alkyl groups) or a hydrogen.

In some examples, the cationic moiety includes any one or more of nitrogen, ammonium, quaternary ammonium, phosphonium, sulfonium, imidazolium, pyridinium, and guanidinium.

In some examples, the substituted pyridine borane complex includes any of the following structures:

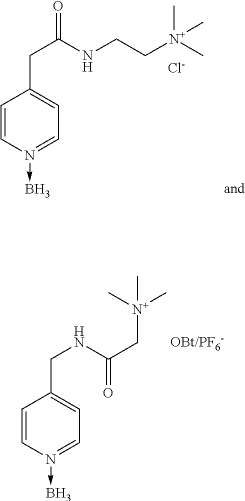

Some examples herein provide a method. The method may include using a ten-eleven translocation (TET) dioxygenase to oxidize any 5-methylcytosine or 5-hydroxymethylcytosine in the polynucleotide to 5-carboxycytosine; using the composition described herein to reduce the 5-carboxycytosine to 5,6-dihydrouracil; and detecting the 5-methylcytosine or 5-hydroxymethylcytosine using the 5,6-dihydrouracil.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates operations in an example workflow for using the present aqueous borane complexes to detect methylcytosine or hydroxymethylcytosine.

FIG. 2 schematically illustrates operations in an example two-step borane reduction procedure.

FIG. 3 provides a graph showing t₅₀ values for the conversion time of a 5caCpG dimer by selected boranes.

FIG. 4 provides a graph showing beta values for caC-modified dsDNA controls using several amine-borane reagents, using a one-step incubation protocol.

FIG. 5 provides a graph showing beta values for human, caC-modified dsDNA controls using several amine-borane reagents, using a two-step incubation protocol.

FIG. 6 shows a schematic of an example interaction between a cationic moiety of a borane and a DNA phosphate backbone.

FIG. 7 provides a graph showing t₅₀ values for the conversion time of a 5caCpG dimer, a 5caC-containing 7-mer and a 5caC-containing 20-mer, using boranes with cationic moieties.

### DETAILED DESCRIPTION

Examples provided herein are related to compositions that include aqueous boranes and polynucleotides. Methods and for using the compositions to detect methylcytosine or hydroxymethylcytosine also are disclosed.

For example, as provided herein, methylcytosine (mC) or hydroxymethylcytosine (hmC) in a polynucleotide may be detected using a workflow in which the mC or hmC is enzymatically or chemically oxidized to carboxycytosine (caC) or formylcytosine (fC), and a borane provided herein is used to reduce the caC or fC to dhU. The polynucleotide then is amplified using polymerase chain reaction (PCR), the dhU is amplified as thymine (T) and as such the mC and hmC are sequenced as T. In comparison, the unmethylated C is amplified, and sequenced, as C. Thus, any Cs in the sequence may be identified as corresponding to C because they had not been converted to T, while any mC or hmC in the sequence may be identified as corresponding to mC or hmC because they had been converted to T. Such a scheme may be referred to as a “four-base” sequencing scheme because any unmethylated C is sequenced as C, providing the ability to obtain both sequence and methylation information from the processed polynucleotide. As provided herein, the present borane complexes are sufficiently water-soluble and mild as to be included in aqueous solutions that may be used to reduce caC or fC in polynucleotides in a practical commercial implementation, and substantially without damaging the polynucleotides thus improving yield and accuracy of detecting mC and hmC while preserving the polynucleotide sequence itself as well.

First, some terms used herein will be briefly explained. Then, some example compositions and example methods using the compositions will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to +10%, such as less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the phrase “generic linker” refers to any molecule or moiety which is capable of coupling, or couples, one element to another. For example, a generic linker may couple an organic molecule to a functional group.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “methylcytosine” or “mC” refers to cytosine that includes a methyl group (—CH₃ or -Me). The methyl group may be located at the 5 position of the cytosine, in which case the mC may be referred to as 5mC.

As used herein, a “derivative” of methylcytosine refers to methylcytosine having an oxidized methyl group. A nonlimiting example of an oxidized methyl group is hydroxymethyl (—CH₂OH), in which case the mC derivative may be referred to as hydroxymethylcytosine or hmC. Another nonlimiting example of an oxidized methyl group is formyl group (—CHO) in which case the mC derivative may be referred to as formylcytosine or fC. Another nonlimiting example of an oxidized methyl group is carboxyl (—COOH), in which case the mC derivative may be referred to as carboxycytosine or caC. The oxidized methyl group may be located at the 5 position of the cytosine, in which case the hmC may be referred to as 5hmC, the fC may be referred to as 5fC, or the caC may be referred to as 5caC. The fC optionally may be present in an acetal form (—CH(OH)₂). The CaC optionally may be present in a salt form (—COO⁻)

As used herein, the term “borane” refers to a chemical compound including BH₃.

As used herein, the term “amine borane complex” refers to a chemical compound that includes a borane which is bonded to a nitrogen within a heterocyclic organic molecule. The heterocyclic organic molecule optionally may include one or more additional heterocyclic atoms besides the nitrogen which is bonded to the borane. In various examples, the heterocyclic organic molecule may include a substituted pyridine, an azole, a pyrimidine, or a pyrazine. As such, the amine borane complex may include a substituted pyridine borane complex, an azole borane complex, or a pyrimidine borane complex.

As used herein, the term “substituted pyridine borane complex” refers to a compound having the structure:

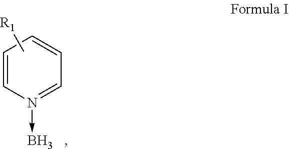

Where the R₁ group may include at least one atom other than a hydrogen. In some examples, the R₁ may be neither a hydrogen nor a methyl group. In some examples, the R₁ group may include a heteroatom. The R₁ group may include any of a sulfur, a nitrogen, a carbonyl group, or a carbon chain. The R₁ group may enhance bond strength between the nitrogen and borane relative to the bond strength of nitrogen and borane in pyridine borane and relative to the bond strength of nitrogen and borane in picoline borane.

As used herein, the term “azole borane complex” refers to a compound having the structure:

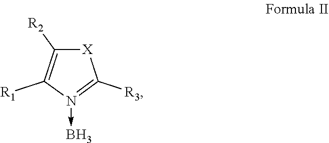

where the X can be S, O, or NR₄ and the R₁, R₂, and R₃ can independently include at least one of H, C, O, N, or S.

As used herein, the term “pyrimidine borane complex” refers to a compound having the structure:

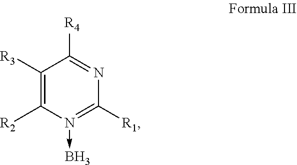

Where R₁, R₂, R₃, and R₄ can independently include at least one of C, O, N, and S.

As used herein, the term “electron donating group” is intended to refer to a group that releases electron density from itself to adjacent atoms, thereby increasing the electron density of the adjacent atoms.

As used herein, the term “electron withdrawing group” is intended to refer to a group that draws electron density from adjacent atoms to itself, thereby reducing electron density of the adjacent atoms.

As used herein, the term “aqueous solution” is intended to refer to any solution in which water functions as a solvent.

Compositions that Include Aqueous Boranes and Polynucleotides, and Methods of Using the Same to Detect Methylcytosine and Hydroxymethyl Cytosine

Some examples provided herein relate to a polynucleotide in contact with an aqueous solution. The aqueous solution may include any of the complexes described herein. Methods of using the present aqueous solutions and complexes for detecting methylcytosine or hydroxymethylcytosine also are described.

In some examples, the complexes and compositions described herein are used to detect methylation of DNA, including detection of methylcytosine and/or hydroxymethylcytosine. In some examples, of the present complexes and compositions are used in a workflow which is some regards similar to the TAPS (TET-assisted pyridine borane sequencing) workflow described in the following references, the entire contents of both of which are incorporated by reference herein: Liu et al., “Bisulfite-free direct detection of 5-methylcytosine at base resolution,” Nature Biotechnology 37:424-429 (2019); Liu et al., “Accurate targeted long-read DNA methylation and hydroxymethylation sequencing with TAPS,” Genome Biology 21: Article no. 54 (2020); Liu et al., “Subtraction-free and bisulfite-free specific sequencing of 5-methylcytosine and its oxidized derivatives at base resolution,” Nature Communications 12: Article no. 618 (2021); and International Publication No. WO 2019/136413 to Song et al. Briefly, and as described in these references, the TAPS workflow uses a ten-eleven translocation (TET) dioxygenase to oxidize 5-methylcytosine (mC) and 5-hydroxymethylcytosine (hmC) in a polynucleotide to 5-carboxycytosine (caC). A pyrimidine borane or picoline borane complex then is used to reduce the 5-carboxycytosine to 5,6-dihydrouracil (dhU). The 5,6-dihyrouracil is sequenced as a T to detect locations where 5mC or 5hmC had been located.

The present inventors have recognized that the previously known TAPS workflow presents several challenges which may impede practical commercial implementation. For example, reduction of 5-carboxycytosine using the pyridine borane complex requires a long incubation time (e.g., about 16 hours) at low pH, high temperature, and a high concentration of reagent (e.g., about 1 M) in order to be efficient. It is believed that these reaction conditions may cause considerable degradation of the DNA, reducing reaction yield and particularly degrading heavily methylated regions. Additionally, the pyridine borane complex is highly toxic and volatile, and requires the use of specialized equipment (such as a fume hood) which may not be compatible with automated sample preparation as may be desirable for use in a commercial implementation. The picoline borane complex also is believed not to be suitable for commercial implementation for similar reasons.

The present inventors have recognized that certain aqueous borane complexes other than those disclosed in the above-cited references suitably may be used to reduce caC or fC to dhU with a higher efficiency, and with less damage to the polynucleotide, than may be achieved using pyridine borane or picoline borane such as used in the TAPS workflow. The present borane complexes are sufficiently water-soluble, reactive, and non-volatile as to be useful at mild pH and without the need for a fume hood or extended reaction times. Without wishing to be bound by any theory, it is believed that by suitably selecting substituent(s) on the present borane complexes, one can fine-tune the reactivity of the complex in such a manner as to enhance reduction of fC or 5caC while reducing DNA damage. Illustratively, without wishing to be bound by any theory, it is believed that in at least some examples of the present borane complexes the enhanced performance, relative to pyridine borane and picoline borane, derives at least in part from an enhanced bond strength between the nitrogen and the borane relative to a bond strength of nitrogen and borane in pyrimidine borane and relative to a bond strength of nitrogen and borane in picoline borane. Without wishing to be bound by any theory, the enhanced bond strength is believed to make the present borane complexes less reactive than pyridine borane and picoline borane, and therefore less damaging to the DNA while providing sufficient activity to reduce caC or fC to dhU. Additionally, the present borane complexes may include substituents selected to provide sufficient solubility and stability in water to form aqueous solutions that may be used to reduce caC or fC to dhU in a polynucleotide under reaction conditions that substantially do not degrade the polynucleotide. As such, the present borane complexes may be readily implemented in a practical commercial setting, and are expected to provide higher reaction yield and improved accuracy in detecting methylcytosine and hydroxymethyl cytosine.

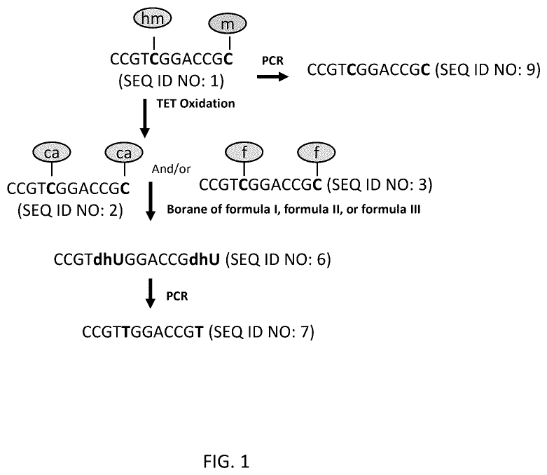

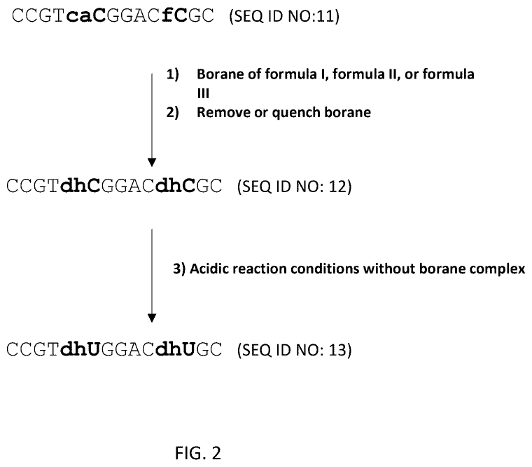

For example, FIG. 1 schematically illustrates operations in an example workflow for using any of the present aqueous borane complexes to detect methylcytosine or hydroxymethylcytosine. The workflow (method) includes using a ten-eleven translocation (TET) dioxygenase to oxidize any 5-methylcytosine or 5-hydroxymethylcytosine in the polynucleotide to 5-carboxycytosine or 5-formylcytosine. Illustratively, in the nonlimiting example shown in FIG. 1, the polynucleotide has the sequence CCGThmCGGACCGmC (SEQ ID NO: 1), and TET dioxygenase is used to oxidize the hmC and mC to caC in a manner similar to that described in the above-cited references, yielding the sequence CCGTcaCGGACCGcaC (SEQ ID NO: 2) and/or to oxidize the hmC and mC to fC, yielding the sequence CCGTfCGGACCGfC (SEQ ID NO: 3). In some examples (not specifically shown in FIG. 2), after use of TET dioxygenase the polynucleotide may include a mixture of caC and fC (e.g., may include the sequence CCGTfCGGACCGcaC (SEQ ID NO: 4) or CCGTcaCGGACCGfC (SEQ ID NO: 5)). A composition provided herein then is used to reduce the fC and/or caC to 5,6-dihydrouracil. In some examples, the composition includes an aqueous solution including a borane complex provided herein, e.g., a borane of formula I, formula II, or formula III such as disclosed in greater detail elsewhere herein. Illustratively, in the nonlimiting example shown in FIG. 1, the aqueous borane complex is used to reduce the caC in the sequence CCGTcaCGGACCGcaC (SEQ ID NO: 2) to dhU, yielding the sequence CCGTdhUGGACCGdhU (SEQ ID NO: 6) and/or to reduce the fC in the sequence CCGTfCGGACCGfC (SEQ ID NO: 3) to dhU, yielding the sequence CCGTdhUGGACCGdhU (SEQ ID NO: 6). In examples including combinations of fC and caC, both similarly may be reduced to dhU.

The mC or hmC then may be detected using the dhU. For example, as illustrated in FIG. 1, a first set of PCR reactions then may be performed on the product of the reduction reaction to generate amplicons of such product. In such amplicons, the dhU (resulting from TET oxidation and subsequent reduction using the present borane complexes) is amplified as T, illustratively yielding the sequence CCGTTGGACCGT (SEQ ID NO: 7) (and complementary sequence GGCAACCTGGCA (SEQ ID NO: 8). Additionally, a second set of PCR reactions may be performed on a separate aliquot of the unreacted polynucleotide. In such amplicons, the mC and hmC are amplified as C, illustratively yielding the sequence CCGTCGGACCGC (SEQ ID NO: 9) (and complementary sequence GGCAGCCTGGCG (SEQ ID NO: 10). The locations in the target polynucleotide at which mC and hmC were located and at which dhU was generated using oxidation and reduction, may be determined by comparing the sequence of the amplicons from the first set of PCR reactions to the sequence of amplicons from the second set of PCR reactions. Bases that are T (or A) in the amplicons from the first set of PCR reactions and that are C (or G) in the amplicons from the second set of PCR reactions may be identified as corresponding to mC and/or hmC because they were chemically converted using TET oxidation and borane reduction.

In some examples, any of the compositions or complexes described herein may be used in a one-step borane reduction procedure such as illustrated in FIG. 1. Illustratively, the polynucleotide including caC or fC resulting from reaction with TET dioxygenase may be contacted with one of the present aqueous compositions (e.g., including a borane of formula I, II, or III) for a sufficient amount of time for the borane to substantially convert the caC or fC into dhU, e.g., for a period of several hours. In one purely illustrative example of such a one-step borane reduction procedure, the polynucleotide including caC or fC may be contacted with the aqueous composition at a pH of about 4-5 at a temperature of about 30-60° C. for about 2-24 hours.

In other examples, any of the compositions or complexes described herein may be used in a two-step borane reduction procedure, as shown in FIG. 2. The two-step borane reduction procedure may be used in place of the one-step borane reduction procedure described above. FIG. 2 schematically illustrates operations in an example two-step borane reduction procedure. In a first step of the borane reduction procedure, the polynucleotide including caC or fC resulting from reaction with TET dioxygenase may be contacted with one of the present aqueous compositions (e.g., including a borane of formula I, II, or III) for a sufficient amount of time for the borane to substantially convert the caC into 5,6-dihydrocytidine (dhC), e.g., for a period of less than that used for the one-step borane procedure and at milder reaction conditions than those described for the one-step borane procedure, e.g., at approximately room temperature to approximately 40° C. at a pH between about 4.5 and 7. Illustratively, in the nonlimiting example shown in FIG. 2, the aqueous borane complex is used to reduce the caC and fC in the sequence CCGTcaCGGACfCGC (SEQ ID NO: 11) to dhC, yielding the sequence CCGTdhCGGACdhCGC (SEQ ID NO: 12). The borane then is removed, for example using a quencher (e.g., alpha-ketoglutarate) and/or by purifying the polynucleotide. In the second step of the borane reduction procedure, the polynucleotide including dhC is subjected to reaction conditions that convert the dhC to dhU in the absence of the borane compound which was used in the first step, illustratively a pH of about 4-5 at a temperature of about 30-70° C. for about 10-24 hours. Illustratively, in the nonlimiting example shown in FIG. 2, acidic reaction conditions (pH<7) are used to convert the dhC in the sequence CCGTdhCGGACdhCGC (SEQ ID NO: 12) to dhU, yielding the sequence CCGTdhUGGACdhUC.

In some examples, any of the borane reduction procedures describe herein utilize a borane with a cationic moiety. In some examples, a borane with a cationic moiety is used in any of the one-step borane reduction procedures described herein. In some examples, a borane with a cationic moiety is used in any of the two-step borane reduction procedures described herein. In some examples, the electrostatic attraction between the positively charged cationic moiety on the borane and the negatively charged DNA phosphate backbone, facilitates an interaction between the cationic moiety on the borane and the DNA phosphate backbone.

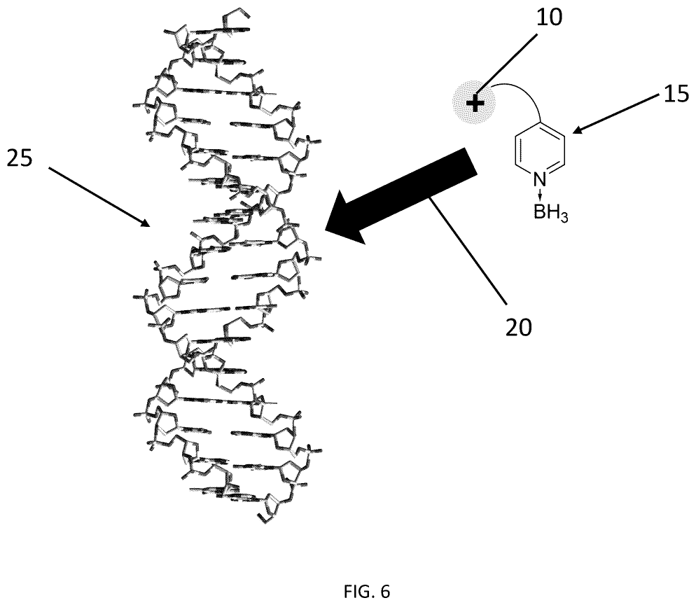

An example of an interaction between a cationic moiety of a borane and a DNA phosphate backbone is shown in FIG. 6. A cationic moiety 10 on a borane 15 is capable of an ion-ion interaction 20 with a DNA phosphate backbone 25. For example, the cationic moiety 10 may be electrostatically attracted to the DNA phosphate backbone 25, which may bring the borane 15 into sufficient proximity to the DNA phosphate backbone to enhance the rate of reaction between the borane and any methylcytosine or hydroxymethylcytosine in the DNA.

In some examples, the presence of the cationic moiety on the borane reduces the required concentration of the borane that is necessary to facilitate an interaction with a DNA phosphate backbone. In some examples, the required concentration of a borane containing a cationic moiety necessary to facilitate an interaction with a DNA phosphate backbone is less than about 40 mM, less than about 35 mM, less than about 30 mM, less than about 25 mM, or less than about 10 mM.

Further details regarding the present compositions and complexes now will be provided.

In some examples, the complex described herein is an amine borane complex. In some examples, the amine borane complex is provided in an aqueous solution. In some examples, the aqueous solution is contact with a polynucleotide, and optionally may be used to detect methylcytosine or hydroxymethylcytosine in the polynucleotide in a manner such as described with reference to FIG. 1 and FIG. 2.

In some examples, the substituted pyridine borane complex is of formula I:

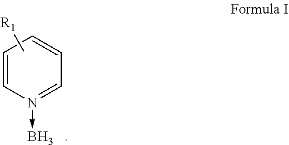

R₁ may include a heteroatom. In some examples, the heteroatom is any of the following elements: iodine, nitrogen, bromine, chlorine, sulfur, phosphorus, fluorine, and oxygen. In some examples, the heteroatom is sulfur, oxygen, or nitrogen.

R₁ may include a functional group. In some examples, the functional group is or includes a carbonyl group. In some examples, the functional group is or includes an alkene group, an alkyne group, an amide group, a sulfur trioxide group, or an aromatic group.

In some examples, R₁ provides enhanced bond strength between the nitrogen and boron, as compared to a bond strength of nitrogen and boron in pyridine borane. In some examples, the bond strength of R₁ between the nitrogen and boron is enhanced at least 50 KJ/mol, at least 100 KJ/mol, at least 150 KJ/mol, at least 200 KJ/mol, at least 250 KJ/mol, at least 300 KJ/mol, at least 350 KJ/mol, at least 400 KJ/mol, at least 450 KJ/mol, at least 500 kJ/mol, at least 550 KJ/mol, at least 600 KJ/mol, at least 650 KJ/mol, at least 700 KJ/mol, at least 750 KJ/mol, at least 800 KJ/mol, at least 850 KJ/mol, at least 900 KJ/mol, at least 950 KJ/mol, or at least 1,000 KJ/mol relative to the bond strength between a nitrogen and boron in pyridine borane. In some examples, the bond strength of R₁ between the nitrogen and boron is enhanced less than 50 kJ/mol relative to the bond strength between a nitrogen and boron in pyridine borane. In some examples, the bond strength of R₁ between the nitrogen and boron is enhanced more than 1,000 KJ/mol relative to the bond strength between a nitrogen and boron in pyridine borane.

In some examples, R₁ provides enhanced bond strength between the nitrogen and boron as compared to bond strength of nitrogen and boron in picoline borane. In some examples, the bond strength of R₁ between the nitrogen and boron is enhanced at least 50 KJ/mol, at least 100 KJ/mol, at least 150 KJ/mol, at least 200 KJ/mol, at least 250 KJ/mol, at least 300 KJ/mol, at least 350 KJ/mol, at least 400 KJ/mol, at least 450 kJ/mol, at least 500 kJ/mol, at least 550 KJ/mol, at least 600 KJ/mol, at least 650 kJ/mol, at least 700 KJ/mol, at least 750 kJ/mol, at least 800 KJ/mol, at least 850 KJ/mol, at least 900 KJ/mol, at least 950 KJ/mol, or at least 1,000 KJ/mol relative to the bond strength between a nitrogen and boron in picoline borane. In some examples, the bond strength of R₁ between the nitrogen and boron is enhanced less than 50 KJ/mol relative to the bond strength between a nitrogen and boron in pyridine borane. In some examples, the bond strength of R₁ between the nitrogen and boron is enhanced more than 1,000 KJ/mol relative to the bond strength between a nitrogen and boron in picoline borane.

In some examples, R₁ is an electron donating group. In some examples, the electron donating group activates the aromatic ring of pyridine. In some examples, the electron donating group is or includes any of the following groups: an oxygen anion, an alcohol group, an alkenyl group, an alkynyl, an aryl group, an amine group, an ether, a thioether, and an alkyl group. Illustratively, in some examples, the electron donating group includes an oxygen that is part of a hydroxide group or a methoxy group.

In some examples, R₁ is an electron withdrawing group. In some examples, the electron withdrawing group deactivates the aromatic ring of pyridine. In some examples, the electron withdrawing group is or includes any of the following groups: a nitro group, an aldehyde group, a ketone group, a carboxylate anion or salt, a cyano group, a carboxylic acid group, an amide group, a carbamate group, a carbonyl group, or an ester group. Illustratively, in some examples, the electron withdrawing group includes a carbonyl group that is part of a group including any of an aldehyde, a ketone, a carboxylic acid, an amide group, a carbamate group, or an ester group.

The electron withdrawing group, electron donating group, or other type of R₁ group may be located at any suitable position in the substituted pyridine borane complex. In some examples, R₁ is ortho to the nitrogen. In some examples, R₁ is meta to the nitrogen. In some examples, R₁ is para to the nitrogen.

In some examples, the composition of the substituted pyridine complex includes any of the following structures:

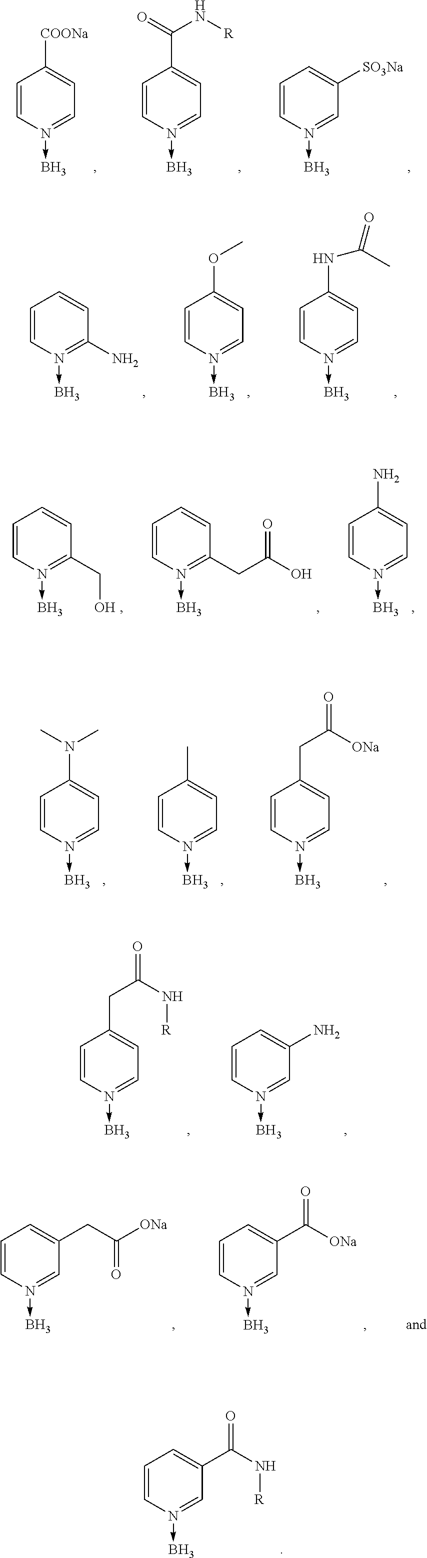

In some examples, the substituted pyridine complex is disubstituted rather than monosubstituted (e.g., falls outside of Formula I) and includes one of the following structures:

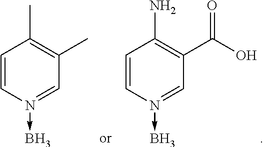

In some examples, the composition of the substituted pyridine complex includes the following structure:

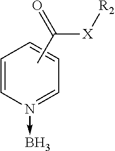

In some examples, X includes O, NH, or S.

In some examples, R₂ includes O, N, or S.

In some examples, R₂ includes an electron donating group. In some examples, the electron donating group is or includes any of the following groups: an oxygen anion, an alcohol group, an amine group, an ether group, a thioether group, an alkenyl group, an alkynyl group, or an alkyl group. In some examples, R₂ includes an electron withdrawing group. In some examples, the electron withdrawing group is or includes any of the following groups: a nitro group, an aldehyde group, a ketone group, a cyano group, a carboxylic acid group, a carbonyl group, an ester group, an amide group, or a carbamate group. In some examples, the electron withdrawing group includes a carbonyl group that is part of a group including any of an aldehyde, a ketone, a carboxylic acid, or an ester.

In some examples, the composition of the substituted pyridine complex includes any of the following structures:

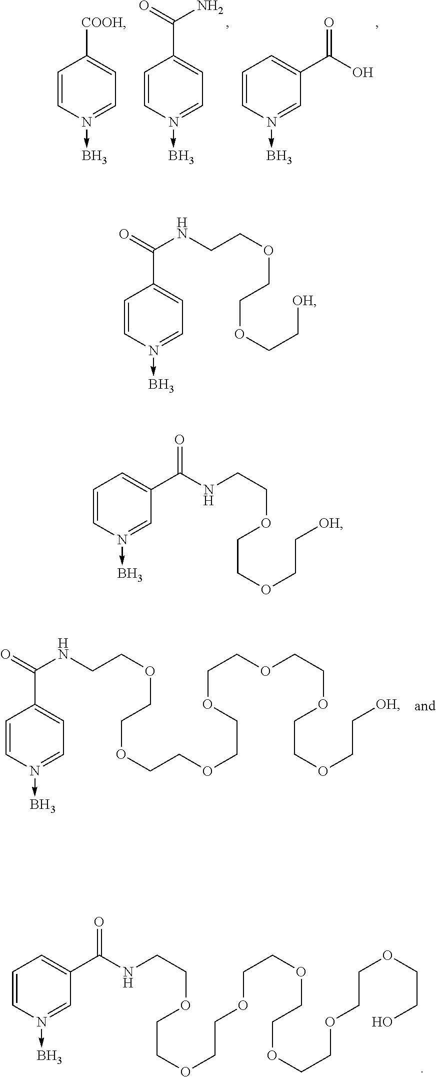

In some examples, the substituted pyridine complex is disubstituted rather than monosubstituted (e.g., falls outside of Formula I) and has the following structure:

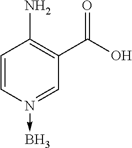

In some examples, the substituted pyridine complex includes the following structure:

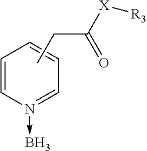

In some examples, X includes O, NH, or S.

In some examples, R₃ includes at least one of O, N, and S.

In some examples, R₃ includes an electron donating group. In some examples, the electron donating group is or includes any of the following groups: an oxygen anion, an alcohol group, an alkenyl group, an alkynyl group, an aryl group, an amine group, an ether group, a thioether group, an alkenyl group, an alkynyl, group, an aryl group, or an alkyl group. In some examples, R₂ includes an electron withdrawing group. In some examples, the electron withdrawing group is or includes any of the following groups: a nitro group, an aldehyde group, a ketone group, a cyano group, a carboxylic acid group, a carbonyl group, an ester group, an amide group, or a carbamate group. In some examples, the electron withdrawing group includes a carbonyl group that is part of a group including any of an aldehyde, a ketone, a carboxylic acid, an ester, an amide group, or a carbamate group.

In some examples, the substituted pyridine complex includes any of the following structures:

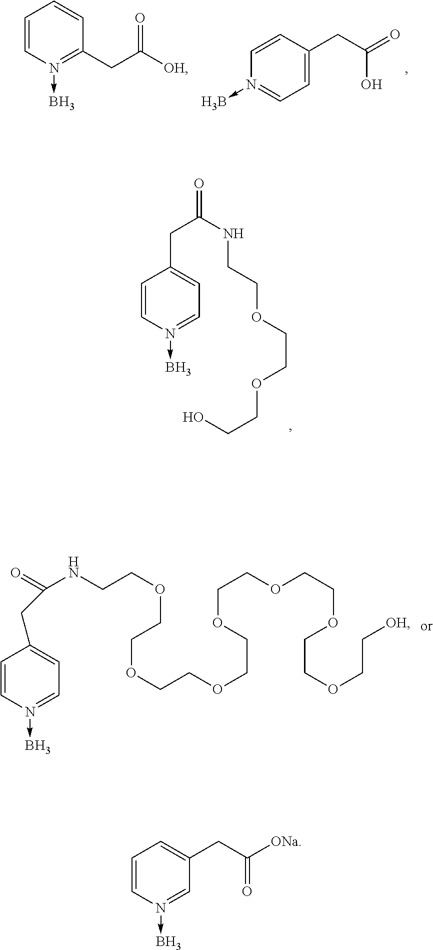

In some examples, the substituted pyridine complex includes the following structure:

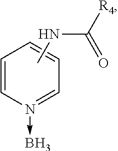

some nonlimiting examples of which include:

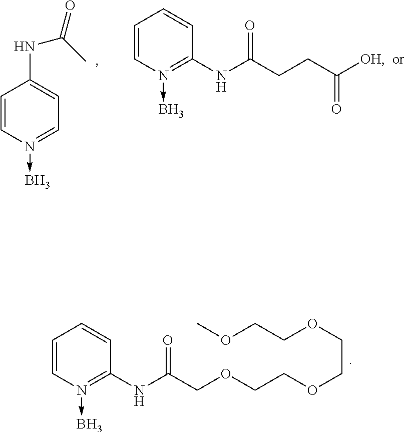

Optionally, R₄ can include a heteroatom (such as N, O, or S) coupled to the carbonyl group.

In some examples a composition including a polynucleotide and an aqueous solution in contact with the polynucleotide is provided. The aqueous solution may include a substituted pyridine borane complex of formula I:

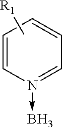

where R₁ includes a cationic moiety.

In some examples, the substituted pyridine borane complex containing a cationic moiety includes any of the following structures:

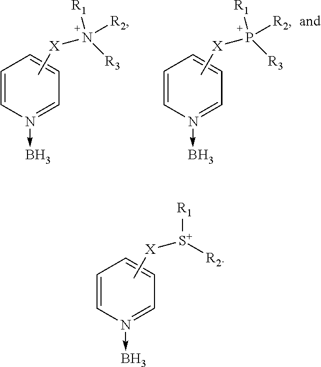

where X is a generic linker group and R₁, R₂, and R₃ include a generic substituent or a hydrogen.

In some examples, the generic substituent includes an alkyl group.

In some examples, the substituted pyridine borane complex containing a cationic moiety includes any of the following structures:

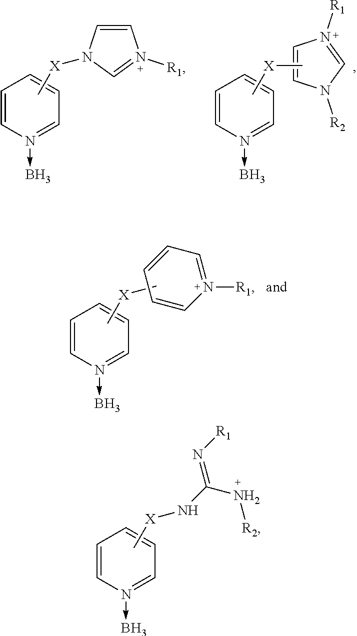

where X is a generic linker group and R₁ and R₂ include a generic substituent or a hydrogen. In some examples, the generic substituent includes an alkyl group.

In some examples, the cationic moiety includes any one or more of nitrogen, ammonium, quaternary ammonium, phosphonium, sulfonium, imidazolium, pyridinium, and guanidinium. In some examples, the cationic moiety includes any metal ion with a net positive charge. In some examples, the metal ion is complexed with a neutral ligand and the neutral ligand is covalently attached to the pyridine ring. In some examples, the cationic moiety includes any ion with a net positive charge.

In some examples, the substituted pyridine borane complex containing a cationic moiety includes any of the following structures:

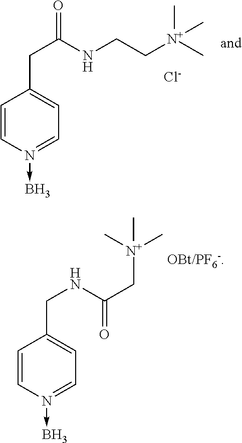

Some examples herein provide a composition including a polynucleotide in contact with an azole borane complex. In some examples, the polynucleotide is in contact with an aqueous solution. In some examples, the composition is used in any workflow described herein, e.g., with reference to FIGS. 1 and 2.

In some examples, the azole borane complex includes the following structure of formula II:

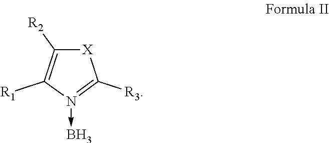

In some examples, X includes S, O, or NR₄. In some examples, X includes nitrogen. In some examples, X includes at least one non-carbon atom.

In some examples, R₁, R₂, and R₃ independently include at least one of H, C, O, N, F, Cl, Br, I, and S.

In some examples, any of R₁, R₂, and R₃ include an electron donating group. In some examples, the electron donating group is or includes any of the following groups: an oxygen anion, an alcohol group, an amine group, an ether group, a thioether group, or an alkyl group. In some examples, any of R₁, R₂, and R₃ include an electron withdrawing group. In some examples, the electron withdrawing group is or includes any of the following groups: a nitro group, an aldehyde group, a ketone group, a cyano group, a carboxylic acid group, a carbonyl group, and an ester group. In some examples, the electron withdrawing group includes a carbonyl group that is part of a group including any of an aldehyde, a ketone, a carboxylic acid, an ester, an amide, or a carbamate.

In some examples, the azole borane complex includes a thiazole complex.

In some examples, the thiazole complex is bonded to one or more carbon chains. In some examples, the one or more carbon chains includes a hydroxide group. In some examples, the one or more carbon chains includes a carbonyl group. In some examples, the thiazole complex is bonded to one or more amino groups.

In some examples, the thiazole complex includes any of the following structures:

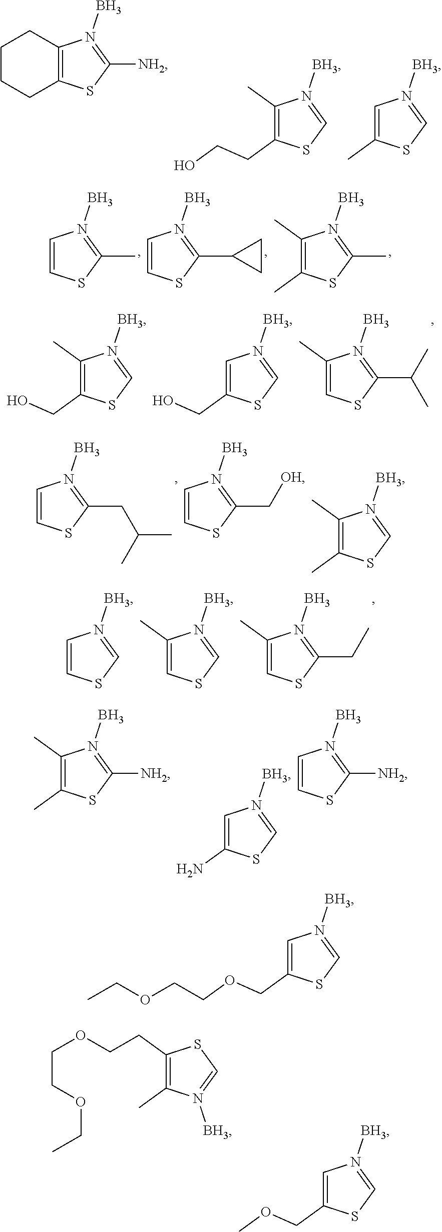

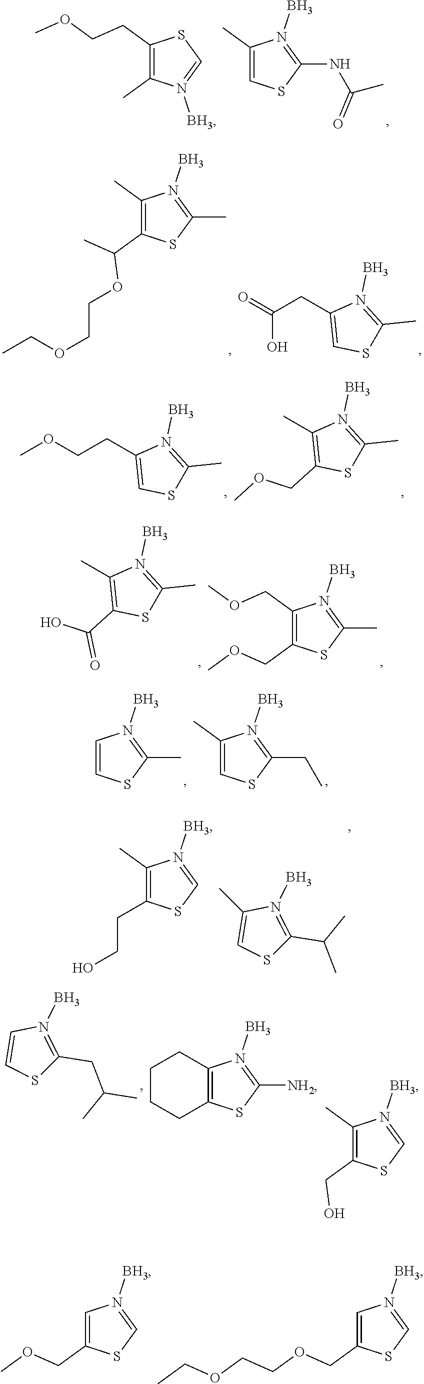

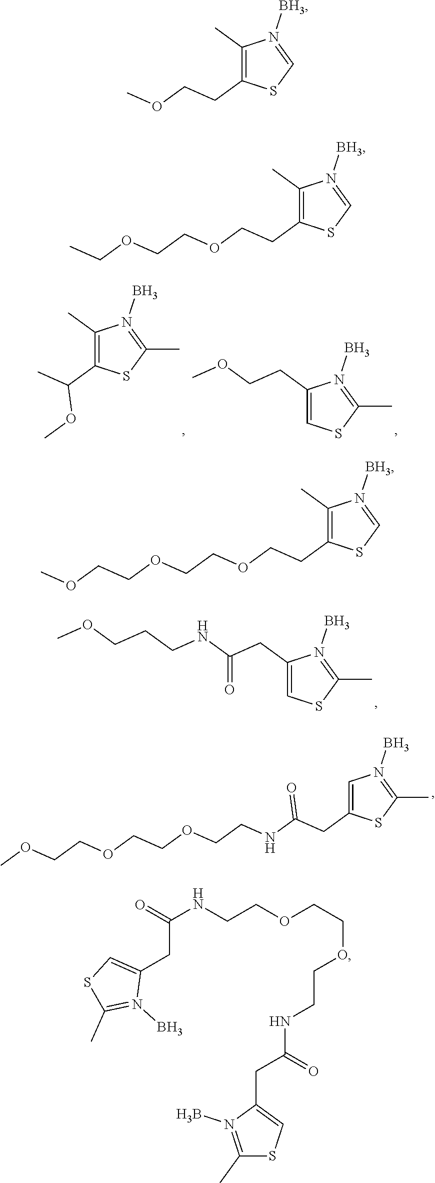

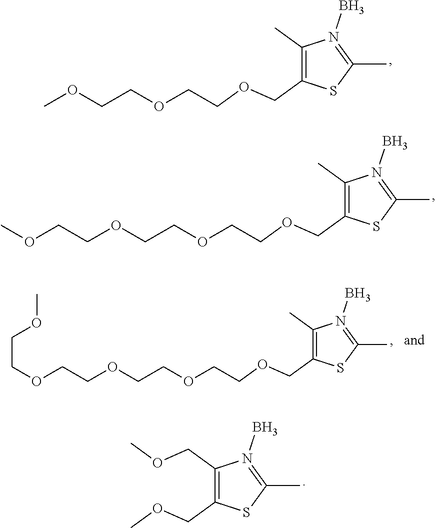

In some examples, the azole borane complex includes an oxazole complex.

In some examples, the oxazole complex is bonded to one or more carbon chains. In some examples, the one or more carbon chains includes a hydroxide group.

In some examples, the oxazole complex includes a formula selected from the group consisting of:

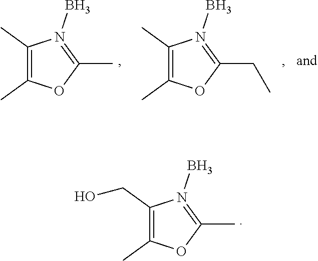

In some examples, the azole borane complex includes an imidazole complex.

In some examples, the imidazole complex includes a formula selected from the group consisting of:

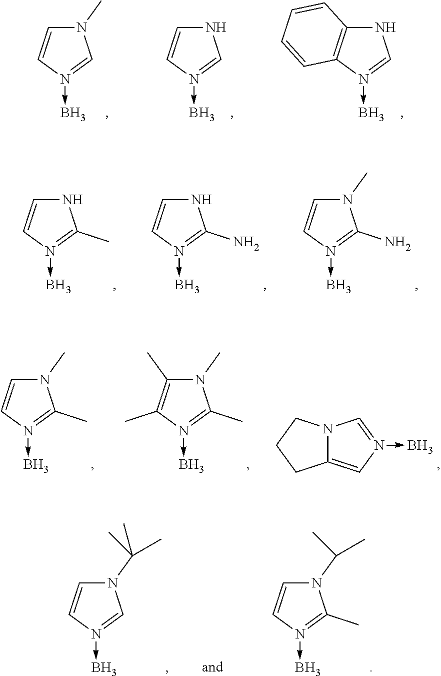

Some examples herein provide a composition including a polynucleotide and pyrimidine borane complex. In some examples, the polynucleotide is in contact with an aqueous solution. In some examples, the composition is used in any workflow described herein, e.g., with reference to FIG. 1 and FIG. 2.

In some examples, the pyrimidine borane complex includes the following structure:

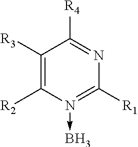

In some examples, R₁, R₂, R₃, and R₄ independently include at least one of C, O, N, and S.

In some examples, any of R₁, R₂. R₃, and R₄ include an electron donating group. In some examples, the electron donating group is or includes any of the following groups: an oxygen anion, an alcohol group, an amine group, an ether group, a thioether group, or an alkyl group. In some examples any of R₁, R₂. R₃, and R₄ include an electron withdrawing group. In some examples, the electron withdrawing group is or includes any of the following groups: a nitro group, an aldehyde group, a ketone group, a cyano group, a carboxylic acid group, a carbonyl group, an ester group, an amide group, or a carbamate group. In some examples, the electron withdrawing group includes a carbonyl group that is part of a group including any of an aldehyde, a ketone, a carboxylic acid, or an ester.

In some examples, the pyrimidine borane complex includes a formula selected from the group consisting of:

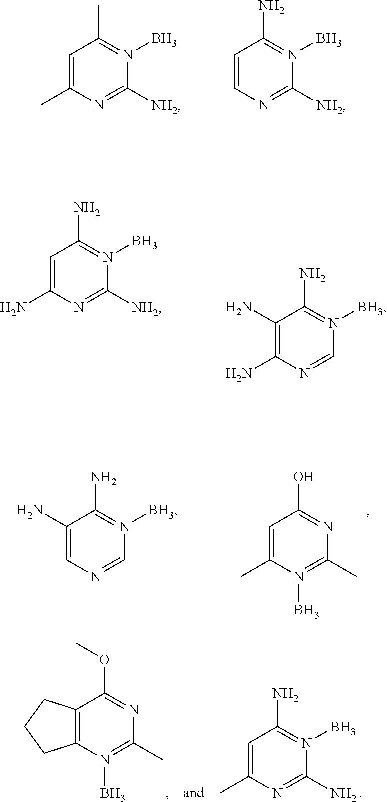

### WORKING EXAMPLES

The following examples are intended to be purely illustrative, and not limiting in any way.

### Example 1: Examples of Chemical Synthesis of Amine-Boranes

### General Procedure 1—Synthesis of Substituted Pyridine Boranes

In a 50 mL flask, sodium hydrogen carbonate (1.68 g, 20 mmol) and an amine (5 mmol) were suspended/dissolved in 10 mL tetrahydrofuran. After addition of H₂O (0.36 mL, 20 mmol), sodium borohydride (0.38 g, 10 mmol) was added slowly in portion, under cooling with an ice bath if necessary to prevent excess heat formation. The mixture was stirred at r.t. overnight. Magnesium sulphate was added, and the mixture filtered through celite. Evaporation of the filtrate provided the product. If containing impurities, this was further purified by flash column chromatography.

### General Procedure 2—Synthesis of Substituted Pyridine Boranes

In a 50 mL flask, sodium hydrogen carbonate (1.68 g, 20 mmol) and an amine or its hydrochloride (5 mmol) were suspended/dissolved in 3 mL dimethylformamide. After addition of H2O (0.36 mL, 20 mmol), sodium borohydride (0.38 g, 10 mmol) was added slowly in portion, under cooling with an ice bath if necessary to prevent excess heat formation. The mixture was stirred at r.t. overnight. 12 ml tetrahydrofuran was added followed by magnesium sulphate and the mixture was filtered through celite. Evaporation of the filtrate provided the product. If containing impurities, this was further purified by flash column chromatography.

### General Procedure 3—Synthesis of Substituted Pyridine Boranes

In a dried flask under N₂, the carboxylic acid (1 equiv.) was dissolved in anhydrous DMF and anhydrous DIPEA (2 equiv.) was added. TSTU (1.3 equiv.) was then added, and the mixture stirred at r.t. for 20-60 min until the activation was complete. The amine (1.3 equiv.) was then added, and the mixture stirred at r.t. overnight. 1 mL H₂O was added, and the solvents were evaporated in vacuo. The crude residue was purified by flash column chromatography to afford a pure product.

### General Procedure 4—Synthesis of Substituted Pyridine Boranes

In a dried flask under N₂, the carboxylic acid (1 equiv.) and the amine (1.3 equiv.) was dissolved in anhydrous DMF and anhydrous DIPEA (2 equiv.) was added. PyBOP (1.3 equiv.) was then added, and the mixture stirred at r.t. overnight. H₂O was added, the aqueous layer was washed with EtOAc three times, and the aqueous layer was evaporated. The crude residue was purified by reverse phase flash chromatography using an increasing gradient of acetonitrile in H₂O to afford a pure product.

### General Procedure 5—Synthesis of Substituted Pyridine Boranes

In a dried flask under N₂, a pyridine derivative (1 equiv.) was dissolved in 9:1 anhydrous DMF/THF and 2,6-lutidine borane (2 equiv., see synthesis below) was added. The mixture was stirred at r.t. overnight. Diethyl ether was then added and the precipitate collected and further washed with diethyl ether, then dried under high vacuum to afford the product.

### Isonicotinic acid borane

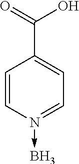

Synthesised according to general procedure 1 from isonicotinic acid in 70% yield.

¹H NMR (400 MHZ, DMSO) δ 8.46 (d, J=5.8 Hz, 1H), 7.88 (d, J=6.5 Hz, 1H), 2.90-2.18 (br, 3H). ¹¹B NMR (128 MHz, DMSO) δ −12.20 (br).

### Nicotinic acid borane

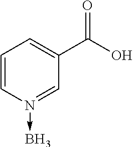

Synthesised according to general procedure 1 from nicotinic acid in 99% yield.

¹H NMR (400 MHZ, DMSO) δ 8.86 (s, 1H), 8.50 (d, J=5.7 Hz, 1H), 8.42 (dd, J=7.7, 1.7 Hz, 1H), 7.63 (dd, J=7.8, 5.6 Hz, 1H), 3.00-2.01 (br, 3H). ¹¹B NMR (128 MHz, DMSO) δ −11.82.

### 2-aminopyridine borane

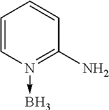

Synthesised according to general procedure 1 from 2-aminopyridine in 99% yield.

¹H NMR (400 MHZ, DMSO) δ 7.92 (dd, J=6.4, 1.8 Hz, 1H), 7.62 (ddd, J=8.7, 6.9, 1.8 Hz, 1H), 6.87 (s (br), 2H), 6.80 (dd, J=8.6, 1.2 Hz, 1H), 6.60 (ddd, J=7.3, 6.2, 1.3 Hz, 1H), 2.04 (d (br), J=113.8 Hz, 3H). ¹¹B NMR (128 MHz, DMSO) δ −16.61.

### 3-aminopyridine borane

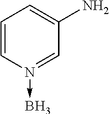

Synthesised according to general procedure 1 from 3-aminopyridine in 99% yield.

¹H NMR (400 MHZ, DMSO) δ 7.86 (d, J=2.5 Hz, 1H), 7.66 (d, J=5.4 Hz, 1H), 7.29 (dd, J=8.4, 5.4 Hz, 1H), 7.18 (ddd, J=8.3, 2.6, 1.1 Hz, 1H), 5.96 (s (br), 2H), 2.70-1.96 (br, 3H). ¹¹B NMR (128 MHz, DMSO) δ −11.86.

### 4-aminopyridine borane

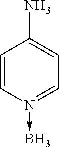

Synthesised according to general procedure 1 from 4-aminopyridine in 99% yield

¹H NMR (400 MHZ, DMSO) δ 7.82 (d, J=7.1 Hz, 2H), 7.03 (s (br), 2H), 6.69-6.41 (m, 2H), 2.60-1.84 (br, 3H). ¹¹B NMR (128 MHz, DMSO) δ −12.94.

### 2-hydroxymethylpyridine borane

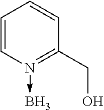

Synthesised according to general procedure 1 from 2-pyridinemethanol in 58% yield.

¹H NMR (400 MHZ, DMSO) δ 8.63 (d, J=5.9 Hz, 1H), 8.19 (t, J=7.8 Hz, 1H), 7.91 (d, J=8.0 Hz, 1H), 7.55 (t, J=6.7 Hz, 1H), 5.82 (t, J=5.6 Hz, 1H), 4.78 (d, J=5.6 Hz, 2H), 2.71-1.91 (br, 3H). ¹¹B NMR (128 MHz, DMSO) δ −14.37.

### 2-pyridineacetic acid borane

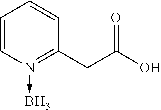

Synthesised according to general procedure 2 from 2-pyridineacetic acid hydrochloride in 35% yield.

¹H NMR (400 MHZ, DMSO) δ 8.67 (dd, J=5.9, 1.6 Hz, 1H), 8.11 (td, J=7.8, 1.7 Hz, 1H), 7.68 (dd, J=7.9, 1.5 Hz, 1H), 7.54 (ddd, J=7.5, 5.8, 1.5 Hz, 1H), 4.07 (s, 2H), 2.68-2.07 (m, 3H). ¹¹B NMR (128 MHz, DMSO) δ −13.23.

### 3-pyridineacetic acid borane

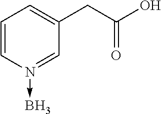

Synthesised according to general procedure 2 from 3-pyridineacetic acid hydrochloride in 52% yield.

¹H NMR (400 MHZ, DMSO) δ 8.49 (d, J=1.9 Hz, 1H), 8.43 (d, J=5.6 Hz, 1H), 8.01 (d, J=7.9 Hz, 1H), 7.63 (dd, J=7.9, 5.7 Hz, 1H), 3.68 (s, 2H), 2.86-2.04 (br, 3H). ¹¹B NMR (128 MHz, DMSO) δ −11.74.

### 4-pyridineacetic acid borane

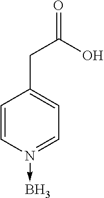

Synthesised according to general procedure 2 from 4-pyridineacetic acid hydrochloride in 29% yield.

¹H NMR (400 MHZ, DMSO) δ 8.48 (d, J=6.2 Hz, 1H), 7.59 (d, J=6.6 Hz, 1H), 3.76 (s, 1H), 2.81-2.10 (m, 4H). ¹¹B NMR (128 MHz, DMSO) δ −11.95.

### PEG₃-isonicotinamide borane

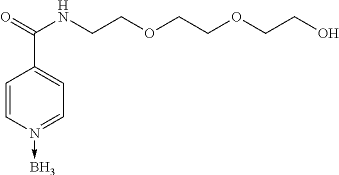

Synthesised according to general procedure 3 from isonicotinic acid borane in 17% yield.

¹H NMR (400 MHZ, CDCl₃) δ 8.64 (d, J=6.4 Hz, 2H), 7.88 (d, J=6.8 Hz, 2H), 7.41 (s, 1H), 3.71-3.56 (m, 12H), 2.97-2.18 (br, 3H). ¹¹B NMR (128 MHz, DMSO) δ −11.41.

### PEG₈-isonicotinamide borane

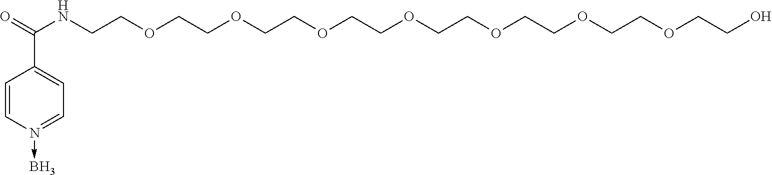

Synthesised according to general procedure 3 from isonicotinic acid borane in 12% yield.

¹H NMR (400 MHZ, DMSO) δ 9.11 (t, J=5.6 Hz, 1H), 8.73 (d, J=6.3 Hz, 2H), 8.03 (d, J=6.7 Hz, 2H), 4.56 (t, J=5.5 Hz, 1H), 3.60-3.37 (m, 32H). ¹¹B NMR (128 MHz, DMSO) δ −11.80.

### PEG₃-nicotinamide borane

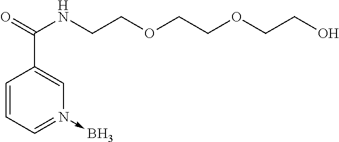

Synthesised according to general procedure 3 from nicotinic acid borane in 29% yield.

¹H NMR (400 MHZ, CDCl₃) δ 9.11 (t, J=1.4 Hz, 1H), 8.71 (d, J=5.6 Hz, 1H), 8.50 (d, J=7.7 Hz, 1H), 7.69 (s, 1H), 7.62 (ddd, J=8.0, 5.7, 0.8 Hz, 1H), 3.91-3.60 (m, 12H), 3.17-1.57 (br, 3H). ¹¹B NMR (128 MHz, DMSO) δ −11.87.

### PEG₃-nicotinamide borane

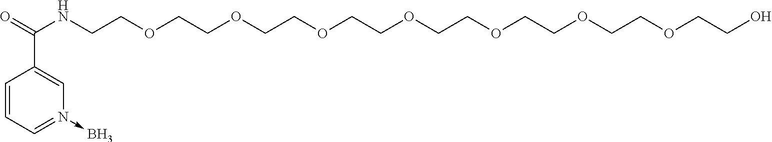

Synthesised according to general procedure 3 from isonicotinic acid borane in 24% yield.

¹H NMR (400 MHz, DMSO) δ 9.06 (t, J=5.5 Hz, 1H), 9.01-8.94 (m, 1H), 8.72 (d, J=5.7 Hz, 1H), 8.53 (d, J=8.0 Hz, 1H), 7.83 (dd, J=8.0, 5.7 Hz, 1H), 4.56 (t, J=5.5 Hz, 1H), 3.67-3.39 (m, 32H). ¹¹B NMR (128 MHz, DMSO) δ −11.45.

### PEG₃-4-pyridineacetamide borane

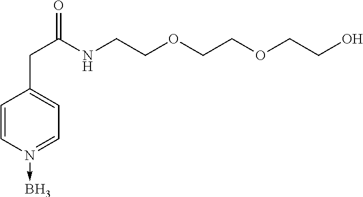

Synthesised according to general procedure 3 from 4-pyridineacetic acid borane.

¹H NMR (400 MHZ, DMSO) δ 8.48 (d, J=6.3 Hz, 2H), 8.30 (s, 1H), 7.56 (d, J=6.4 Hz, 2H), 4.58 (t, J=5.4 Hz, 1H), 3.65 (s, 2H), 3.50-3.37 (m, 10H), 3.22 (q, J=5.6 Hz, 2H). ¹¹B NMR (128 MHz, DMSO) δ −11.53.

### PEG₈-4-pyridineacetamide

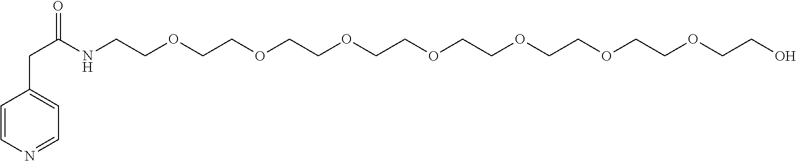 [SMILES: O=C(Cc1ccncc1)NCCOCCOCCOCCOCCOCCOCCOCCO]

Synthesised according to general procedure 3 from 4-pyridineacetic acid in 47% yield.

¹H NMR (400 MHZ, DMSO) δ 8.48 (d, J=6.1 Hz, 2H), 8.27 (t, J=5.7 Hz, 1H), 7.29 (d, J=6.1 Hz, 2H), 4.58 (s, 1H), 3.53-3.28 (br, 32H).

### PEG₈-4-pyridineacetamide borane

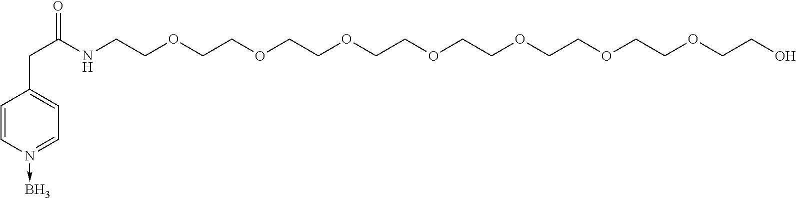

Synthesised according to general procedure 1 from PEG₈-4-pyridineacetamide in 44% yield.

¹H NMR (400 MHZ, DMSO) δ 8.48 (d, J=6.4 Hz, 2H), 8.31 (t, J=5.6 Hz, 1H), 7.57 (d, J=6.7 Hz, 2H), 4.57 (t, J=5.4 Hz, 1H), 3.65 (s, 2H), 3.54-3.38 (m, 32H). ¹¹B NMR (128 MHz, DMSO) δ −11.82.

### 4MeP: 4-Methylpyridine borane

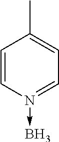

Synthesised according to general procedure 1 from 4-methylpyridine in 99% yield.

¹H NMR (400 MHZ, DMSO) δ 8.42 (d, J=6.6 Hz, 2H), 7.62-7.38 (m, 2H), 2.94-1.93 (m, 6H). ¹¹B NMR (128 MHz, DMSO) δ −11.98.

### 4HMP: 4-Hydroxymethylpyridine borane

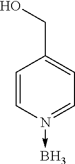

Synthesised according to general procedure 1 from 4-pyridinemethanol in 99% yield.

¹H NMR (400 MHZ, DMSO) δ 8.64-8.41 (m, 2H), 7.61 (dt, J=6.6, 0.9 Hz, 2H), 5.69 (t, J=5.7 Hz, 1H), 4.66 (d, J=4.9 Hz, 2H), 2.87-2.00 (br, 3H). ¹¹B NMR (128 MHZ, DMSO) δ −12.01.

### 4-Pyridinepropionic acid borane

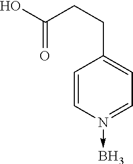

Synthesised according to general procedure 1 from 4-pyridinepropionic acid in 51% yield.

¹H NMR (400 MHZ, DMSO) δ 8.36 (d, J=6.9 Hz, 2H), 7.53 (d, J=6.6 Hz, 2H), 2.86 (t, J=7.2 Hz, 2H), 2.20 (t, J=7.2 Hz, 5H). ¹¹B NMR (128 MHz, DMSO) δ −12.12.

### 4HEP: 4-(2-Hydroxyethyl)pyridine borane

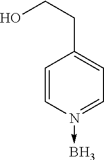

Synthesised according to general procedure 1 from 2-(pyridine-4-yl) ethanol in 99% yield.

¹H NMR (400 MHZ, DMSO) δ 8.53-8.34 (m, 2H), 7.66-7.42 (m, 2H), 4.78 (t, J=5.2 Hz, 1H), 3.69 (td, J=6.2, 5.2 Hz, 2H), 2.86 (t, J=6.2 Hz, 2H), 2.78-2.02 (m, 3H). ¹¹B NMR (128 MHz, DMSO) δ −12.08.

### 4HPP: 4-(3-Hydroxypropyl)pyridine borane

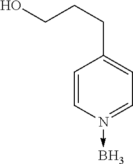

Synthesised according to general procedure 1 from 2-(pyridine-4-yl) propanol in 99% yield.

¹H NMR (400 MHZ, DMSO) δ 8.43 (d, J=6.7 Hz, 2H), 7.55 (d, J=6.6 Hz, 2H), 4.57 (t, J=5.1 Hz, 1H), 3.41 (td, J=6.3, 5.1 Hz, 2H), 2.77 (dd, J=8.8, 6.7 Hz, 2H), 2.67-2.03 (br, 3H), 1.75 (ddt, J=9.1, 7.7, 6.3 Hz, 2H). ¹¹B NMR (128 MHz, DMSO) δ −11.92.

### 2,6-Lutidine borane

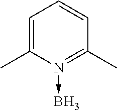

Synthesised according to general procedure 1 from 2,6-lutidine in 45% yield.

¹H NMR (400 MHz, DMSO) δ 7.89 (t, J=7.8 Hz, 1H), 7.50 (d, J=7.8 Hz, 2H), 2.67 (d, J=0.8 Hz, 6H), 2.62-1.67 (br, 3H). ¹¹B NMR (128 MHz, DMSO) δ −17.57.

### 4-(Betainamidomethyl)pyridine (OBt/PF6 salt)

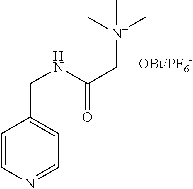

Synthesised according to general procedure 4 from betaine and 4-(aminomethyl)pyridine in 37% yield.

¹H NMR (400 MHZ, DMSO) δ 10.06 (s, 1H), 8.48 (d, J=6.1 Hz, 2H), 7.27 (d, J=6.1 Hz, 2H), 4.39 (d, J=5.8 Hz, 2H), 4.31 (s, 2H), 3.24 (s, 9H) (counterion signals not reported).

### 4AMP-Bet: 4-(Betainamidomethyl)pyridine borane (OBt/PF6 salt)

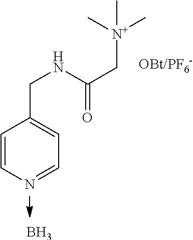

Synthesised according to general procedure 5 from 4-Aminopyridine betaine amide (HOBt/PF6 salt) in 63% yield.

¹H NMR (400 MHZ, DMSO) δ 9.17 (t, J=5.9 Hz, 1H), 8.59-8.50 (m, 2H), 7.63-7.52 (m, 2H), 4.51 (d, J=5.9 Hz, 2H), 4.22 (s, 2H), 3.23 (s, 9H), 2.85-2.27 (m, 3H) (counterion signals not reported). ¹¹B NMR (128 MHz, DMSO) δ −11.74.

### 2-aminoethyltrimethylammonium 4-pyridineacetamide (chloride salt)

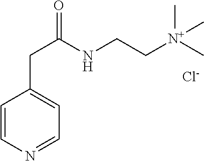 [SMILES: C[N+](C)(C)CCNC(=O)Cc1ccncc1 ; [Cl-]]

Synthesised according to general procedure 4 from 4-Pyridineacetic acid hydrochloride and 2-aminoethyltrimethylammonium chloride hydrochloride in 91% yield.

¹H NMR (400 MHZ, DMSO) δ 9.19 (s, 1H), 8.71 (d, J=6.5 Hz, 2H), 7.75-7.70 (m, 2H), 3.79 (s, 2H), 3.51 (t, J=6.1 Hz, 2H), 3.42 (dd, J=6.8, 5.4 Hz, 2H), 3.12 (s, 9H).

### 4PA-CholA: 2-aminoethyltrimethylammonium 4-pyridineacetamide borane (chloride salt)

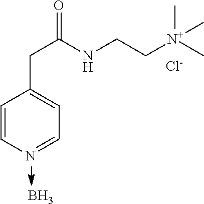

Synthesised according to general procedure 5 from 2-aminoethyltrimethylammonium 4-pyridineacetamide in 19% yield.

¹H NMR (400 MHZ, DMSO) δ 8.84 (d, J=6.8 Hz, 1H), 8.50 (d, J=6.2 Hz, 2H), 7.60 (d, J=6.6 Hz, 2H), 3.73 (s, 2H), 3.50 (t, J=6.3 Hz, 2H), 3.37 (t, J=6.6 Hz, 2H), 3.09 (s, 9H), 2.62-2.28 (m, 9H). ¹¹B NMR (128 MHz, DMSO) δ −12.24.

### Example 2: Examples of Chemical Synthesis of Thiazole Boranes, Oxazole Boranes, Pyrimidine Boranes, and Imidazole Boranes

### General Procedure 6—Synthesis of Thiazole Boranes, Oxazole Boranes, Pyrimidine Boranes, And Imidazole Boranes

Sodium borohydride (1.5 eq.) and powdered sodium bicarbonate (3 eq.) were transferred to an appropriate oven-dried round bottom flask and charged with a magnetic stir-bar. The corresponding N-heterocycle (5 mmol in a typical experiment) was charged into the reaction flask followed by addition of reagent-grade tetrahydrofuran (1.9 mL per 5 mmol of N-heterocycle) at room temperature. Under vigorous stirring, 1.9 mL of 14.4% v/v solution water in THF was added dropwise. After 16 h, the reaction mixtures were filtered through sodium sulfate and celite and the solid residue washed with THF. Removal of the solvent under reduced pressure from the filtrate yielded a crude material, which was purified by flash chromatography (20% PET-EtOAc/35% PET-EtOAc). Typical yields were 30-80%.

Each of the thiazole boranes, oxazole boranes, pyrimidine boranes, and imidazole boranes shown below were synthesized using General Procedure 6.

### VM81: thiazole borane

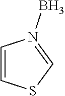

¹H NMR (400 MHZ, DMSO) δ 9.44 (s, 1H), 8.03 (dd, J=3.4, 2.4 Hz, 1H), 7.96 (dd, J=3.5, 1.1 Hz, 1H), 2.8-1.8 (br., 3H). ¹¹B NMR (DMSO, 128 MHZ): −15.67 ppm.

### VM117: 5-methylthiazole borane

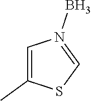

¹H NMR (400 MHZ, DMSO) δ 9.22 (d, J=1.3 Hz, 1H), 7.71 (dt, J=1.3 Hz, 1H), 2.47 (d, J=1.2 Hz, 3H), 2.9-1.8 (br, 3H). ¹¹B NMR (DMSO, 128 MHz): −15.7.

### VM118: 2,4,5-trimethyloxazole borane

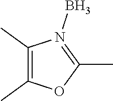

¹H NMR (400 MHZ, DMSO) δ 2.55 (s, 3H), 2.27 (s, 3H), 2.5-1.7 (br, 3H), 2.06 (d, J=1.2 Hz, 3H). ¹¹B NMR (DMSO, 128 MHz): −21.64 ppm.

### VM133: 2-methylthiazole borane

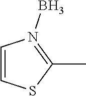

¹H NMR (400 MHZ, DMSO) δ 7.80 (d, J=3.8 Hz, 1H), 7.75 (d, J=3.8 Hz, 1H), 2.73 (s, 3H), 2.6-1.7 (br, 3H). ¹¹B (DMSO, 128 MHz): −17.44 ppm.

### VM134: 2,4,5-trimethylthiazole borane

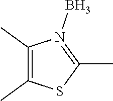

¹H NMR (400 MHz, DMSO) δ 2.69 (s, 3H), 2.35 (s, 3H), 2.5-1.5 (br., 3H), 2.29 (d, J=0.9 Hz, 3H). ¹¹B (DMSO, 128 MHz): −19.4 ppm.

### VM135: 2-ethyl-4-methylthiazole borane

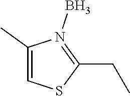

¹H NMR (400 MHZ, DMSO) δ 7.51 (d, J=1.1 Hz, 1H), 3.14 (q, J=7.4 Hz, 2H), 2.6-1.5 (br., 3H) 2.39 (d, J=1.1 Hz, 3H), 1.30 (t, J=7.4 Hz, 3H). ¹¹B (DMSO, 128 MHz): −19.6 ppm.

### VM136: 4-methylthiazole borane

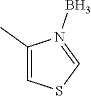

¹H NMR (400 MHZ, DMSO) δ 9.42 (d, J=2.6 Hz, 1H), 7.69 (dd, J=2.7, 1.2 Hz, 1H), 2.7-1.7 (br., 3H), 2.40 (d, J=1.0 Hz, 3H). ¹¹B (DMSO, 128 MHz): − ppm.

### VM137: 5-ethanol-4-methyl thiazole borane

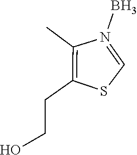

¹H NMR (400 MHZ, DMSO) δ 9.28 (s, 1H), 4.98 (t, J=5.1 Hz, 1H) 3.62 (q, J=5.6 Hz, 2H), 2.94 (t, J=5.9 Hz, 2H), 2.7-1.7 (br., 3H), 2.33 (s, 3H). ¹¹B (DMSO, 128 MHz): −16.4 ppm.

### VM138: 4,5-dimethylthiazole borane

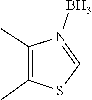

¹H NMR (400 MHz, DMSO): δ 9.25 (s, 1H), 2.41 (s, 3H), 2.32 (s, 3H), 2.6-1.6 (br., 3H). ¹¹B (DMSO, 128 MHz): −19.3 ppm.

### VM139: 2-isopropyl-4-methyl thiazole

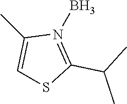

¹H NMR (400 MHZ, DMSO): δ 7.6 (s, 1H), 3.6 (m, 1H), 2.40 (s, 3H), 2.6-1.6 (br., 3H), 1.33 (d, 6H). ¹¹B (DMSO, 128 MHz): −19.2 ppm.

### VM140: 2-Isobutyryl Thiazole Borane

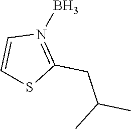

¹H NMR (400 MHZ, DMSO) δ 7.82 (s, 2H), 3.06 (d, J=7.2 Hz, 2H), 2.15 (m, 1H), 0.95 (d, J=6.6 Hz, 6H). ¹¹B (DMSO, 128 MHz): −17.2 ppm.

### VM154: 2-amino-4,5-dimethylthiazole borane

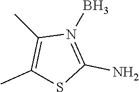

¹H NMR (400 MHZ, DMSO) δ 7.61 (s, 2H), 2.13 (d, J=1.0 Hz, 3H), 2.3-1.3 (br., 3H), 2.06 (d, J=1.0 Hz, 3H). ¹¹B (DMSO, 128 MHz): −19.9 ppm.

### VM160: 5-hydroxymethylthiazole borane

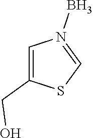

¹H NMR (400 MHZ, DMSO) δ 9.32 (t, J=1.0 Hz, 1H), 7.81 (q, J=1.2 Hz, 1H), 5.89 (t, J=5.8 Hz, 1H), 4.70 (d, J=3.0, 1.1 Hz, 2H), 2.5-1.5 (br., 3H). ¹¹B (DMSO, 128 MHz): −15.7 ppm.

### VM163: 2-hydroxymethyl thiazole borane

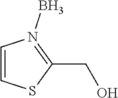

¹H NMR (400 MHZ, DMSO) δ 7.87 (d, J=3.6 Hz, 1H), 7.81 (d, J=3.6 Hz, 1H), 6.03 (t, J=5.8 Hz, 1H), 4.80 (d, J=4.2 Hz, 2H), 2.5-1.5 (br., 3H). ¹¹B (DMSO, 128 MHz): −16.7 ppm.

### VM167: 2-ethyl, 4,5-dimethyl oxazole borane

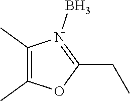

¹H NMR (400 MHZ, DMSO) δ 2.95 (q, J=7.6 Hz, 2H), 2.5-1.5 (br., 3H), 2.28 (d, J=1.1 Hz, 3H), 2.06 (d, J=1.1 Hz, 3H), 1.21 (q, J=7.6 Hz, 3H). ¹¹B (DMSO, 128 MHz): −22.4 ppm.

### VM168: 2-amino-4,5,6,7-tetrahydrobenzothiazole borane

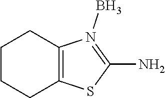

¹H NMR (400 MHZ, DMSO) & 7.63 (s, 2H), 2.45 (m, 4H), 2.4-1.4 (br., 3H) 1.71 (m, 4H). ¹¹B (DMSO, 128 MHz): −22.4 ppm.

### VM197: 1,2,4,5-tetramethylimidazole borane

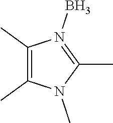

¹H NMR (400 MHZ, DMSO) δ 3.46 (s, 3H), 2.40 (s, 3H), 2.3-1.3 (br., 3H), 2.11 (s, 3H), 2.06 (s, 3H). ¹¹B NMR (128 MHz, DMSO) δ −21.74.

### VM199: 1,2-Dimethylimidazole Borane

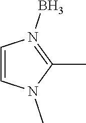

¹H NMR (400 MHZ, DMSO) δ 7.21 (d, J=1.7 Hz, 1H), 6.91 (d, J=1.7 Hz, 1H), 3.62 (s, 3H), 2.5-1.5 (br., 3H), 2.37 (s, 3H). ¹¹B NMR (128 MHz, DMSO) δ −19.22.

### VM200: 5H,6H,7H-pyrrolo[1,2-a]imidazole borane

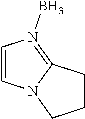

¹H NMR (400 MHZ, DMSO) δ 7.26 (d, J=1.7 Hz, 1H), 6.95 (d, J=1.7 Hz, 1H), 4.09-4.01 (m, 2H), 2.84 (t, J=7.6 Hz, 2H), 2.62-2.50 (m, 6H), 2.5-1.5 (br., 3H) ¹¹B NMR (128 MHz, DMSO) δ −19.31.

### VM202: (4-methyl-1,3-thiazol-5-yl)methanol borane

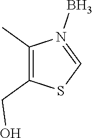

¹H NMR (400 MHZ, DMSO) δ 9.35 (d, J=1.0 Hz, 1H), 5.86 (t, J=5.6 Hz, 1H), 4.70-4.64 (t, 2H), 2.6-1.0 (br., 3H), 2.32 (s, 3H). ¹¹B NMR (128 MHz, DMSO) δ −16.67.

### VM204: 1-(tert-butyl)imidazole borane

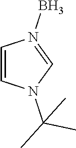

¹H NMR (400 MHZ, DMSO) δ 8.29 (d, J=1.6 Hz, 1H), 7.57 (t, J=1.8 Hz, 1H), 7.04 (t, J=1.6 Hz, 1H), 2.6-1.6 (br., s, 3H), 1.53 (s, 9H). ¹¹B NMR (128 MHz, DMSO) δ −18.45.

### VM205: 2-methyl-1-(propan-2-yl)-imidazole borane

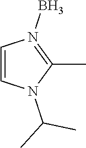

¹H NMR (400 MHZ, DMSO) δ 7.40 (d, J=1.9 Hz, 1H), 6.96 (d, J=1.9 Hz, 1H), 4.47 (hept, J=6.7 Hz, 1H), 2.6-1.6 (br., s, 3H), 2.43 (s, 3H), 1.37 (d, J=6.7 Hz, 6H). ¹¹B NMR (128 MHz, DMSO) δ −19.28.

### VM230: 4-hydroxy 2,6-dimethylpyrimidine borane

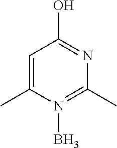

¹H NMR (400 MHZ, DMSO) δ 5.65 (s, 1H), 2.4-1.4 (br., s, 3H), 2.33 (s, 3H), 2.17 (s, 3H). ¹¹B NMR (128 MHz, DMSO) δ −19.36.

### VM253: 5-methoxymethylthiazole borane

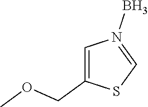

¹H NMR (400 MHZ, DMSO) δ 9.39 (t, J=1.0 Hz, 1H), 7.93 (t, J=1.1 Hz, 1H), 4.66 (d, J=0.9 Hz, 2H), 3.31 (s, 3H), 2.9-1.0 (br., 3H) ¹¹B NMR (128 MHz, DMSO) δ −15.74.

### VM254: 5-(2-ethoxy)ethoxymethylthiazole borane

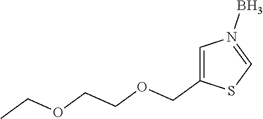

¹H NMR (400 MHZ, DMSO) δ 9.38 (t, J=1.0 Hz, 1H), 7.92 (q, J=1.1 Hz, 1H), 4.74 (d, J=1.0 Hz, 2H), 3.70-3.57 (m, −2H), 3.54-3.48 (m, 2H), 3.43 (q, J=7.0 Hz, 2H), 2.7-1.7 (br., 3H), 1.10 (t, J=7.0, 1.8 Hz, 3H). ¹¹B NMR (128 MHz, DMSO) δ −15.66.

### VM269: 5-methoxyethyl-4-methyl thiazole borane

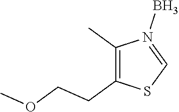

¹H NMR (400 MHZ, DMSO) δ 9.30 (d, J=0.9 Hz, 1H), 3.53 (t, J=5.9 Hz, 2H), 3.28 (s, 3H), 3.03 (t, J=5.9 Hz, 2H), 2.7-1. (br., 3H), 2.33 (s, 3H). ¹¹B NMR (128 MHz, DMSO) δ 16.47.

### VM270: 5-(2-ethoxy)ethoxyethyl-4-methyl thiazole borane

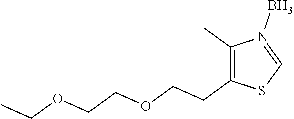

¹H NMR (400 MHZ, DMSO) δ 9.30 (d, J=0.9 Hz, 1H), 3.65-3.36 (m, 8H), 3.03 (t, J=5.9 Hz, 2H), 2.5-1.5 (br., 3H) 1.09 (t, J=7.0 Hz, 3H). ¹¹B NMR (128 MHZ, DMSO) δ 16.43.

### VM284: 2-(2,4-dimethyl-1,3-thiazol-5-yl)-2-methoxyethane borane

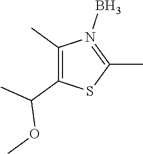

¹H NMR (400 MHZ, DMSO) δ 4.79 (q, J=6.4 Hz, 1H), 3.20 (s, 3H), 2.74 (d, J=0.7 Hz, 3H), 2.5-1.5 (br., 3H), 2.37 (s, 3H), 1.38 (d, J=6.4 Hz, 3H). ¹¹B NMR (128 MHZ, DMSO) δ −19.52.

### VM292: 4-(2-methoxyethyl)-2-methyl-1,3-thiazole borane

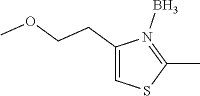

¹H NMR (400 MHZ, DMSO) δ 7.48 (d, J=0.9 Hz, 1H), 3.63 (t, J=6.5 Hz, 2H), 3.26 (s, 3H), 3.06 (td, J=6.5, 0.9 Hz, 2H), 2.74 (d, J=0.6 Hz, 3H)., 2.5-1.5 (br., 3H) ¹¹B NMR (128 MHz, DMSO) δ −19.58.

### VM293: 5-(methoxymethyl)-2,4-dimethyl-1,3-thiazole borane

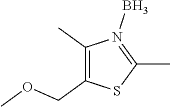

¹H NMR (400 MHZ, DMSO) δ 4.60 (s, 2H), 3.30 (s, 3H), 2.74 (d, J=0.7 Hz, 3H), 2.35 (d, J=0.5 Hz, 3H). ¹¹B NMR (128 MHz, DMSO) δ −19.45.

### VM301: 4-methyl-5-(2-(2-methoxyethoxy)ethoxy)ethyl}-1,3-thiazole

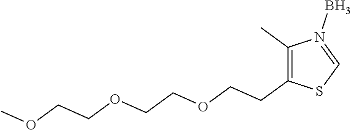

¹H NMR (400 MHZ, DMSO) δ 9.30 (d, J=0.9 Hz, 1H), 3.67-3.56 (m, 2H), 3.56-3.47 (m, 6H), 3.45-3.38 (m, 2H), 3.23 (s, 3H), 3.03 (t, J=5.9 Hz, 2H), 2.5-1.5 (br., 3H), 2.33 (s, 3H). ¹¹B NMR (128 MHz, DMSO) δ −16.49.

### VM326: N-(3-methoxypropyl)-2-(2-methyl-1,3-thiazol-4-yl)acetamide

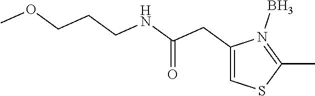

¹H NMR (400 MHZ, DMSO) δ 8.04 (t, J=5.7 Hz, 1H), 7.52 (d, J=0.9 Hz, 1H), 3.71 (d, J=0.8 Hz, 2H), 3.22 (s, 3H), 3.11 (td, J=6.9, 5.6 Hz, 2H), 2.74 (d, J=0.6 Hz, 3H), 2.5-1.5 (br., 3H) 1.65 (p, J=6.6 Hz, 2H). ¹¹B NMR (128 MHz, DMSO) δ −19.63.

### VM331: N-[2-[2-(2-methoxyethoxy)ethoxy]-ethyl]-2-(2-methyl-1,3-thiazol-4-yl)acetamide

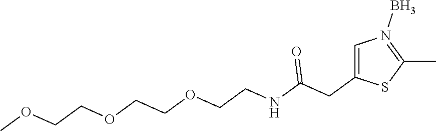

¹H NMR (400 MHZ, DMSO) δ 8.34 (t, J=5.6 Hz, 1H), 7.62 (d, J=1.1 Hz, 1H), 3.76-3.71 (m, 2H), 3.53-3.49 (m, 7H), 3.46-3.36 (m, 4H), 3.28-3.17 (m, 4H), 2.68 (s, 3H), 2.5-1.5 (br., 3H) ¹¹B NMR (128 MHz, DMSO) δ −17.59.

### VM341: Glycol (di-2-(2-(2-methyl-1,3-thiazol-4-yl)acetylamino)ether

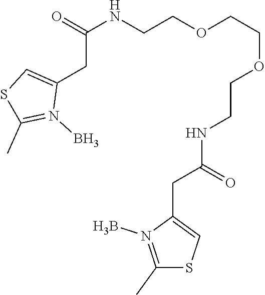

¹H NMR (400 MHZ, DMSO) δ 8.15 (t, J=5.6 Hz, 2H), 7.52 (d, J=0.8 Hz, 2H), 3.73 (d, J=0.9 Hz, 4H), 3.53 (d, J=3.4 Hz, 4H), 3.44 (t, J=5.9 Hz, 4H), 3.24 (q, J=6.1 Hz, 4H), 2.74 (s, 6H), 2.5-1.5 (br., 3H). ¹¹B NMR (128 MHz, DMSO) δ −19.67.

### VM344: 5-(2-(2-methoxyethoxy)ethoxy)methyl-2,4-dimethyl-1,3-thiazole borane

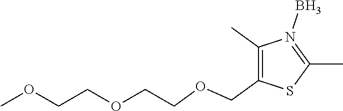

¹H NMR (400 MHZ, DMSO) δ 4.69 (s, 2H), 3.63-3.48 (m, 6H), 3.48-3.40 (m, 2H), 3.24 (s, 3H), 2.74 (s, 3H), 2.5-1.5 (br., 3H), 2.35 (s, 3H). ¹¹B NMR (128 MHz, DMSO) δ 19.44.

### VM356: 5-(2-(2-(2-methoxyethoxy)ethoxy)ethoxy)methyl-2,4-dimethyl-1,3-thiazole borane

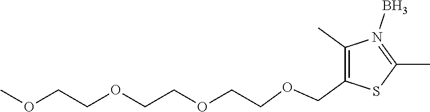

¹H NMR (400 MHZ, DMSO) δ 4.69 (s, 2H), 3.62-3.48 (m, 10H), 3.47-3.39 (m, 2H), 3.24 (s, 3H), 2.74 (s, 3H), 2.5-1.5 (br., 3H) 2.35 (s, 3H). ¹¹B NMR (128 MHz, DMSO) δ 19.46.

### VM357: 5-(2-(2-(2-(2-methoxyethoxy)ethoxy)ethoxy)ethoxy)methyl-2,4-dimethyl-1,3-thiazole borane

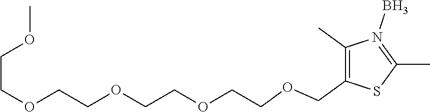

¹H NMR (400 MHZ, DMSO) δ 4.69 (s, 2H), 3.61-3.47 (m, 14H), 3.44-3.39 (m, 2H), 3.23 (s, 3H), 2.74 (d, J=0.7 Hz, 3H), 2.5-1.5 (br., 3H) 2.34 (d, J=0.5 Hz, 3H). ¹¹B NMR (128 MHz, DMSO) δ −19.44.

### VM371: 4,5-di(methoxymethyl)-2-methyl-1,3-thiazole borane

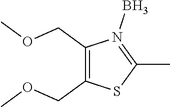

¹H NMR (400 MHZ, DMSO) δ 4.70 (s, 2H), 4.63 (s, 2H), 3.35 (s, 3H), 3.30 (s, 3H), 2.75 (d, J=0.7 Hz, 3H), 2.5-1.5 (br., 3H) ¹¹B NMR (128 MHz, DMSO) δ −19.23.

### VM384: 4-methoxy-2-methyl-6,7-dihydro-5H-cyclopentapyrimidine borane

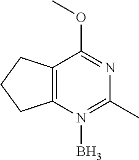

¹H NMR (400 MHZ, DMSO) δ 4.02 (s, 3H), 3.14 (tt, J=8.2, 1.0 Hz, 2H), 2.83 (t, J=7.4 Hz, 2H), 2.78-2.70 (m, 3H), 2.5-1.5 (br., 3H), 2.09 (tt, J=8.4, 7.2 Hz, 2H). ¹¹B NMR (128 MHz, DMSO) δ −17.70.

### N-imidazole borane

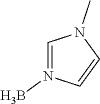

¹H NMR (400 MHZ, DMSO-d₆) δ 8.20 (s, 1H), 7.32 (s, 1H), 7.00 (s, 1H), 3.70 (s, 3H), 1.99 (br s, 3H). ¹¹B NMR (128 MHz, DMSO) δ −18.2.

### Imidazole borane

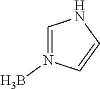

¹H NMR (400 MHz, DMSO-d₆) δ 12.62 (s, 1H), 8.21 (s, 1H), 7.31 (s, 1H), 7.02 (s, 1H) 2.61-1.76 (m, 3H). ¹¹B NMR (128 MHz, DMSO) δ −18.86.

### N-methyl 2-aminoimidazole borane

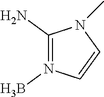

¹H NMR (400 MHZ, DMSO-d₆) δ 6.68 (d, J=2.0 Hz, 1H), 6.42 (d, J=2.1 Hz, 1H), 6.12 (s, 2H), 3.37 (s, 3H), 2.3-1.3 (br s). ¹¹B NMR (128 MHz, DMSO) δ −20.96.

### 2-methylimidazole borane

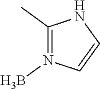

¹H NMR (400 MHZ, DMSO-d₆) δ 7.15 (d, J=1.7 Hz, 1H), 6.91 (d, J=1.7 Hz, 1H), 2.35 (s, 3H), 2.3-1.6 (br s). ¹¹B NMR (128 MHz, DMSO) δ −20.96.

### Example 3: Amine borane reduction kinetics on 5caCpG

To 45 μL of an aqueous solution of 0.556 mM 5caCpG dimer (0.5 mM final concentration) in 556 mM NaOAc buffer (pH 4.3, 500 mM final concentration) were added 5 μL of a stock solution of the respective borane (0.5 M or 1.0 M in DMF, 50 mM or 100 mM final concentration) and the reaction was incubated at 25° C. after short vortexing. Aliquots of 8 μL were taken at set time points and were quenched immediately with 4 μL alpha-ketoglutarate monosodium salt (1.0 M in H₂O) and analysed by RP-UHPLC for consumption of the caCpG starting material and formation of the product DHUpG.

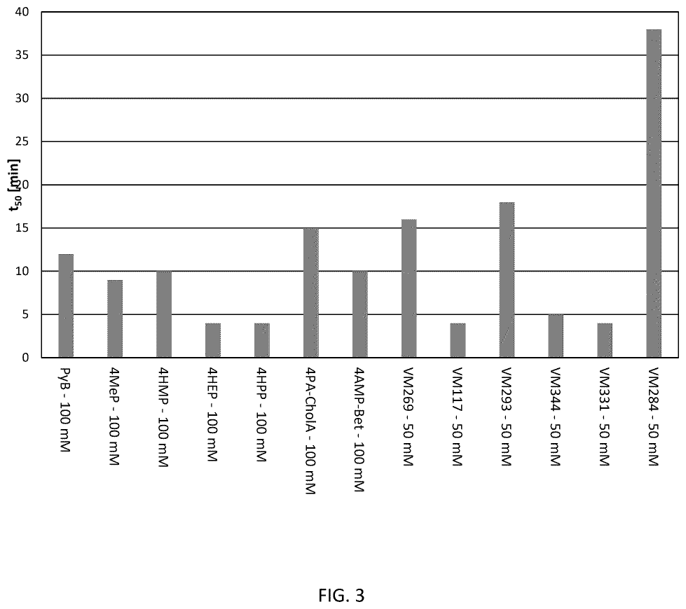

The time required for consumption of 50% of 5caCpG (t₅₀) was calculated from the kinetic curves. FIG. 3 shows t₅₀ values for the reduction time of a 5caCpG dimer, using the selected boranes pyridine borane (PyB), 4MeP, 4HMP, 4HEP, 4HPP, 4PA-CholA, 4AMP-Bet, VM269, VM117, VM293, VM344, VM331, and VM284. The data show efficient conversion kinetics, as each of the tested boranes reduced 50% of the 5caCpG in less than 40 minutes. Most of the tested boranes achieved this conversion in less than 15 minutes. This data shows the effectiveness in using these tested boranes as part of an experimental procedure to detect methylated cytosines.

### Example 4: caC Detection of dsDNA by Illumina Sequencing Using Amine-Boranes

### General Procedure for One-Step Overnight Incubation

An aliquot of 2 M borane stock solution in DMF was added to a buffered solution containing calculated amount of dsDNA (300 bp-500 bp long, containing 4 known caC sites per strand), resulting in the final borane concentration of 100 mM and pH of 4.3 (0.5 M NaOAc). The reaction mixture (50 μL) was shaken at 40° C. for 16 h. Then, the samples were purified by 1.8×SPRI (pH adjusted with 1 M Tris buffer pH=9.0). The purified DNA was PCR-amplified using the NEB-Next Enzymatic Methyl-Seq kit's NEB-Next Q5U Master Mix along with IDT-ILMN NextEra UDI Index primers. Finally, 0.9×SPRI purification was performed. The DNA libraries were normalized to 2 nM and 20 pM sequenced on an Illumina NextSeq instrument. The sequencing data was analysed to extract the beta value, which reflects the percentage conversion of caC to T.

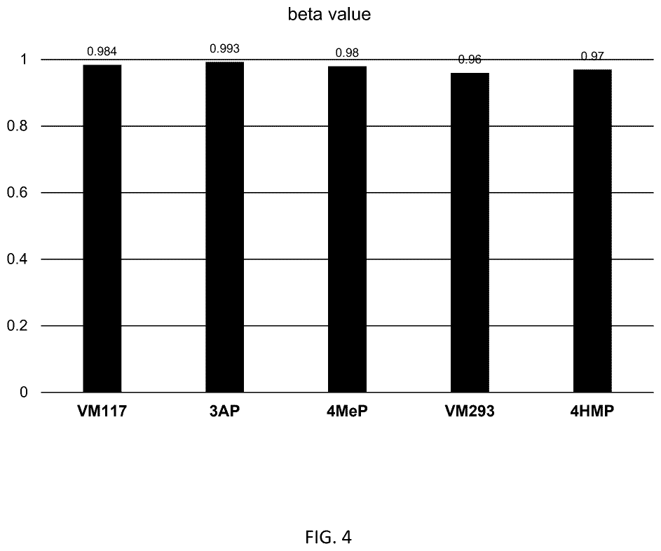

The boranes tested included 5-methylthiazole borane VM117, 3-aminopyridine borane (3 AP), 4-methylpyridine-borane (4MeP), 5-(methoxymethyl)-2,4-dimethyl-1,3-thiazole borane (VM293), and 4-hydroxymethylpyridine borane (4HMP). FIG. 4 shows that these tested boranes, using the one-step incubation protocol described above, displayed a beta value (the percentage of caC sites detected by Illumina sequencing) very close to the value expected for the dsDNA sample used (beta=1). The percentage of caC sites were detected by Illumina sequencing.

This data indicates very efficient conversion caC to DHU by the borane complexes on dsDNA samples. Thus, this data shows that these tested boranes used in a one-step incubation protocol are effective in detecting methylated cytosines.

### General Procedure for Two-Step Incubation

A sample of human DNA NA12878 was treated with human TET2 dioxygenase to oxidise mC to caC. The sample was purified by a 1.8×SPRI and resuspended in RSB buffer. 2 M stocks of the amine-boranes were prepared in DMF, then added to the DNA to a final concentration of 40 mM or 100 mM, along with pH 4.3 sodium acetate buffer to a final concentration of 0.5 M. For the one-step protocol, the samples were incubated at 40° C. for 18 hours and 750 rpm shaking. For the two-step protocol, the samples were incubated at 40° C. for 2.5 hours and 750 rpm shaking, then quenched with a 1 M solution of alpha-ketoglutarate at pH 4.3 (to a final concentration of 400 mM) and kept at 40° C. for 18 hours and 750 rpm shaking. After the incubation, all samples underwent a 1.8×SPRI purification after adding 1 M pH 9 TRIS to basify the pH. Libraries were then amplified via PCR using the NEB-Next Enzymatic Methyl-Seq kit's NEB-Next Q5U Master Mix along with NEBNext Multiplex Oligos for Illumina Index primers. A final 0.9×SPRI was performed, before libraries were normalized to 20 pM sequenced on an Illumina NovaSeq (2×151 cycles). The sequencing data was analysed to extract the beta value, which reflects the percentage conversion of 5mC or 5hmC to T.

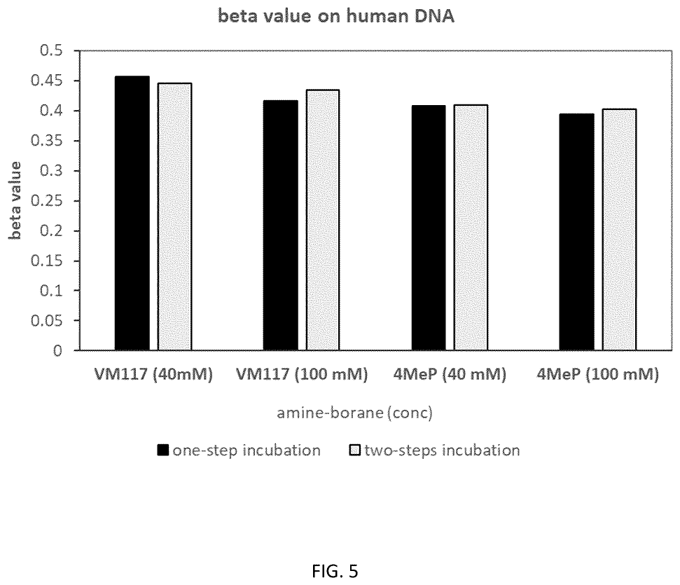

FIG. 5 shows beta values for caC-modified dsDNA controls using the amine-borane reagents VM117 (5-methylthiazole borane) and 4MeP (4-methylpyridine borane) at two different concentrations, using a two-step incubation protocol. The percentage caC sites were detected by Illumina sequencing.

As shown in FIG. 5, the beta values obtained with the two-step protocol are consistent with the value obtained with the one-step protocol. This data indicates that using the boranes VM117 and 4MeP are effective in detecting methylated cytosines, whether a one-step incubation protocol is used or a two-step incubation protocol is used.

### Example 5: Reaction Kinetics of caC Reduction on Nucleotides, Using Boranes with Cationic Moieties

To 45 μL of an aqueous solution of 0.111 mM 5caCpG dimer (0.1 mM final concentration) or 55.6 μM 7mer oligodeoxyribonucleotide (50 μM final concentration, sequence: AT[5caC]GCTA) or 22.2 μM 20mer oligodeoxyribonucleotide (20 μM final concentration, sequence: TTTCAGCTC[5caC]GGTCACGCTC) in 556 mM NaOAc buffer (pH 4.3, 500 mM final concentration) were added 5 μL of a stock solution of the respective borane (100 mM in DMF, 10 mM final concentration) and the reaction was incubated at 25° C. after short vortexing. Aliquots of 8 μL were taken at set time points and were quenched immediately with 4 μL alpha-ketoglutarate monosodium salt (1.0 M in H₂O) and analysed by RP-UHPLC for consumption of the caC moiety in the starting material and formation of the product DHU moiety.

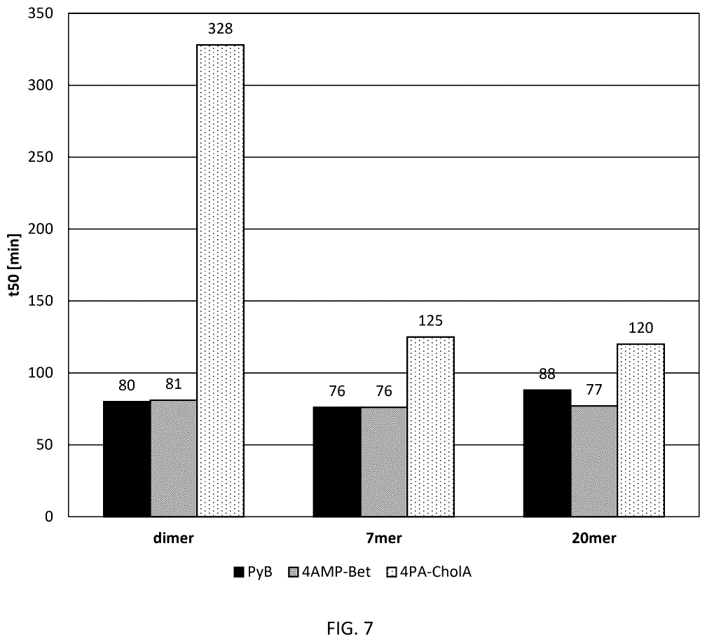

Pyridine borane (PyB) as well as two boranes containing cationic moieties were tested: (i) (4-(Betainamidomethyl)pyridine borane (OBt/PF6 salt) (4-AMP-Bet) and (ii) 2-aminoethyltrimethylammonium 4-pyridineacetamide borane (chloride salt) (4PA-CholA). As shown in FIG. 7, both of the boranes tested containing the cationic moieties (4AMP-Bet and 4PA-CholA) showed efficient reaction kinetics of consumption of caC, on the 7mer and the 20mer. These data show that these boranes containing cationic moieties are effective in detecting methylated cytosines.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A composition, comprising:
a polynucleotide; and
an aqueous solution in contact with the polynucleotide, the aqueous solution comprising a substituted pyridine borane complex of formula I: 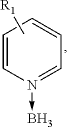
wherein R₁ comprises a heteroatom; or
wherein R₁ comprises any one of a sulfur, an oxygen, a nitrogen, a carbonyl group, and a carbon chain; or
wherein R₁ provides an enhanced bond strength between the nitrogen and boron (i) as compared to a bond strength of nitrogen and boron in pyridine borane and (ii) as compared to a bond strength of nitrogen and boron in picoline borane; or
wherein R₁ comprises a cationic moiety.

2. The composition of claim 1, wherein R₁ comprises the heteroatom and the heteroatom comprises at least one of iodine, nitrogen, bromine, chlorine, fluorine, sulfur, phosphorus, and oxygen.

3. The composition of claim 1
wherein R₁ comprises any one of the sulfur, the oxygen, the nitrogen, the carbonyl group, and the carbon chain.

4. The composition of claim 1
wherein R₁ provides the enhanced bond strength between the nitrogen and boron (i) as compared to the bond strength of nitrogen and boron in pyridine borane and (ii) as compared to the bond strength of nitrogen and boron in picoline borane.

5. The composition of claim 1, wherein R₁ is an electron donating group or an electron withdrawing group.

6. The composition of claim 5, wherein the electron donating group comprises an oxygen that is bonded to the pyridine ring, or a nitrogen that is bonded to the pyridine ring.

7. The composition of claim 6 wherein the oxygen is part of a hydroxide group or methoxy group.

8. (canceled)

9. The composition of claim 6, wherein the nitrogen is part of an amino group, an amide group, or a carbamate group.

10. (canceled)

11. The composition of claim 5, wherein the electron withdrawing group comprises a carbonyl group that is bonded to the pyridine ring, a carboxylate, or a carboxamide.

12. The composition of claim 11, wherein the carbonyl group is part of a group comprising any of an aldehyde, a ketone, a carboxylic acid, an ester, or an amide group.

13. (canceled)

14. The composition of claim 1, wherein R₁ is ortho to the nitrogen, meta to the nitrogen, or para to the nitrogen.

15. (canceled)

16. (canceled)

17. The composition of claim 1, wherein the pyridine borane complex has a structure selected from the group consisting of:

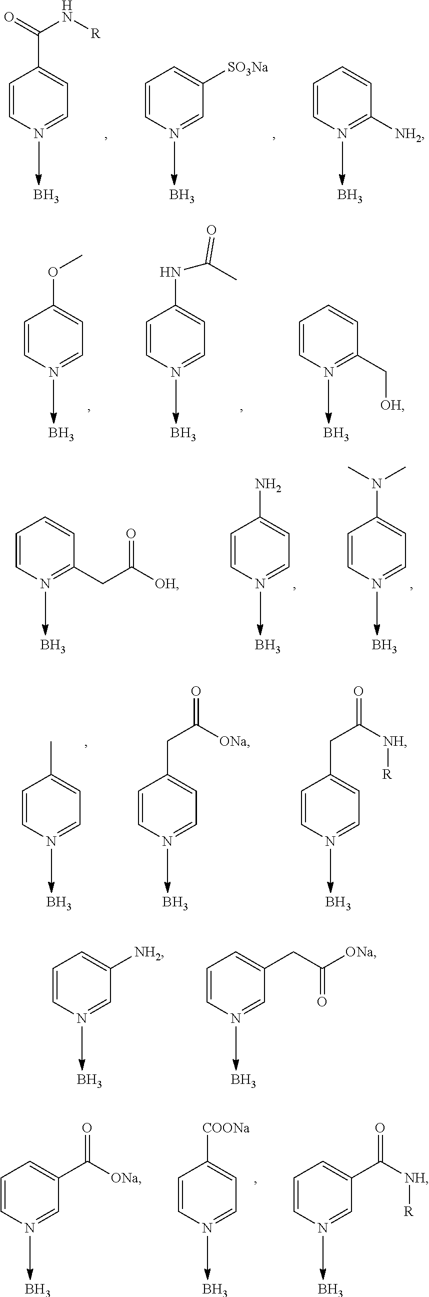

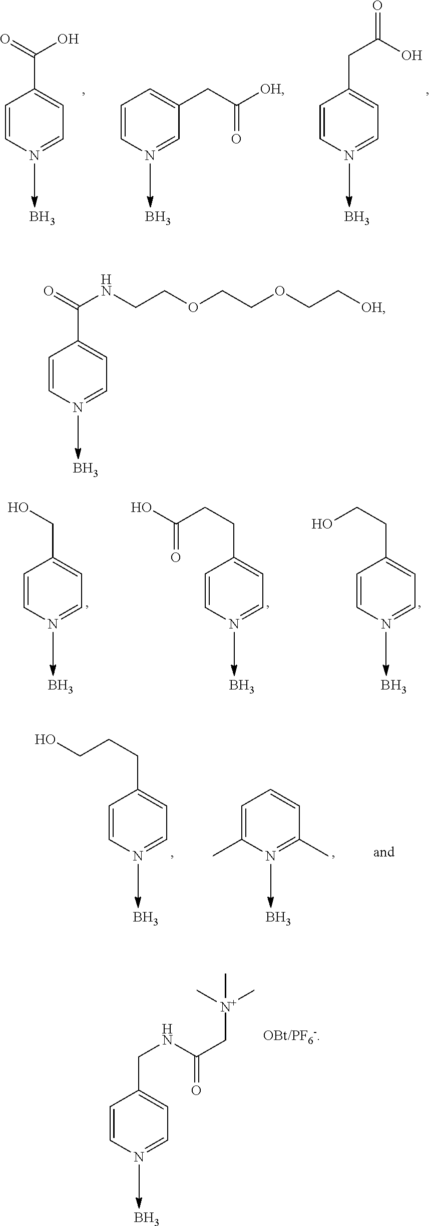

18. The composition of claim 1, wherein the pyridine borane complex is of formula Ia: 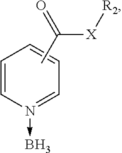
where X is O, NH, or S, and wherein R₂ comprises at least one of O, N, and S.

19. The composition of claim 18, wherein the pyridine borane complex of formula Ia has a structure selected from the group consisting of:

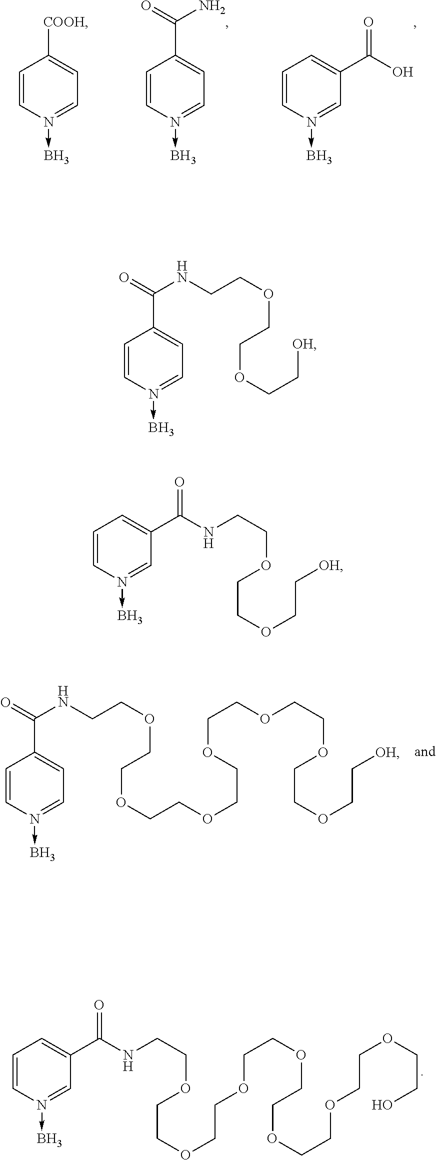

20. The composition of claim 1, wherein the pyridine borane complex is of formula Ib: 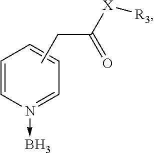
where X is O, NH, or S, and wherein R₃ comprises at least one of O, N, and S.

21. The composition of claim 20, wherein the pyridine borane complex of formula Ib has a structure selected from the group consisting of:

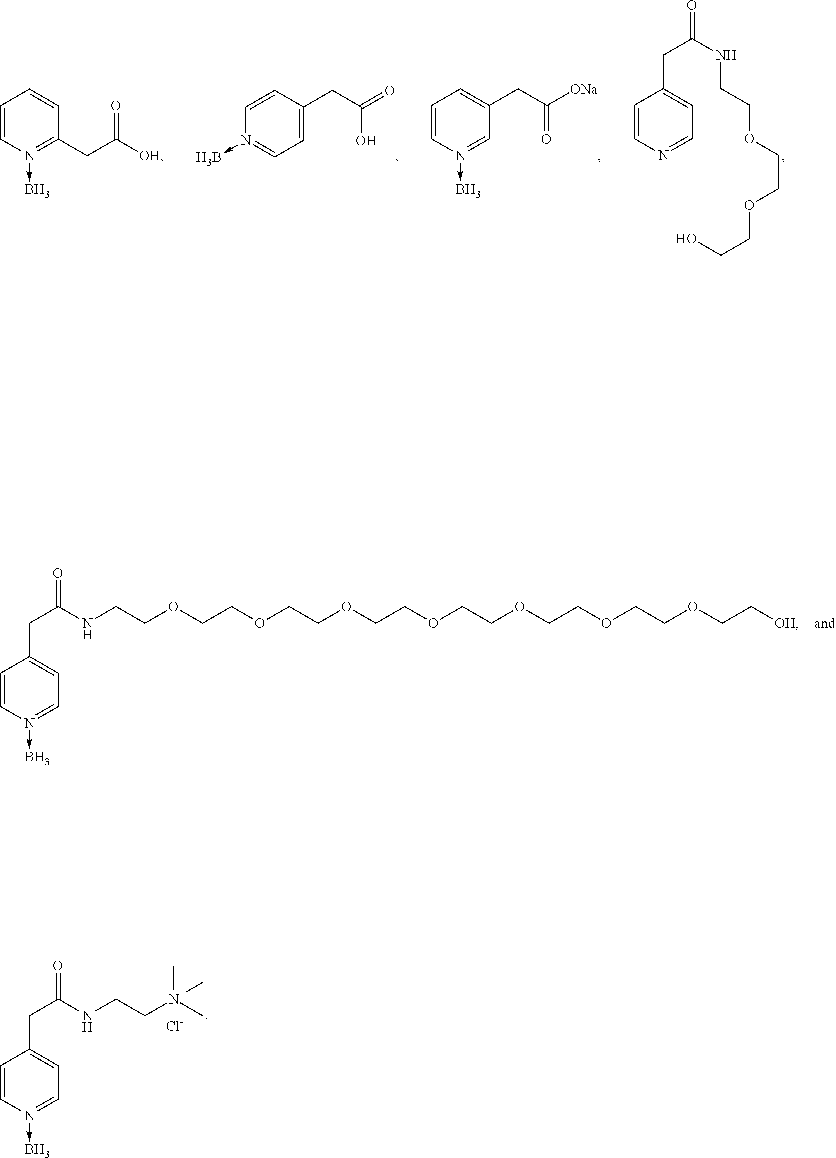

22. A composition, comprising:
a polynucleotide; and
an aqueous solution in contact with the polynucleotide, the aqueous solution comprising an azole borane complex of formula II: 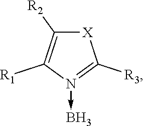
wherein X is S, O, or NR₄; and
wherein R₁, R₂, R₃, and R₄ independently comprise at least one of H, C, O, N, and S.

23. (canceled)

24. (canceled)

25. (canceled)

26. (canceled)

27. (canceled)

28. (canceled)

29. A composition, comprising:
a polynucleotide; and
an aqueous solution in contact with the polynucleotide, the aqueous solution comprising a pyrimidine borane complex of formula III: 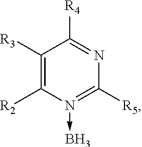
wherein R₁, R₂, and R₃ independently comprise at least one of C, O, N, and S.

30. (canceled)

31. The composition of claim 1,
wherein R₁ comprises the cationic moiety.

32. The composition of claim 31, wherein the substituted pyridine borane complex comprises any of the following structures: 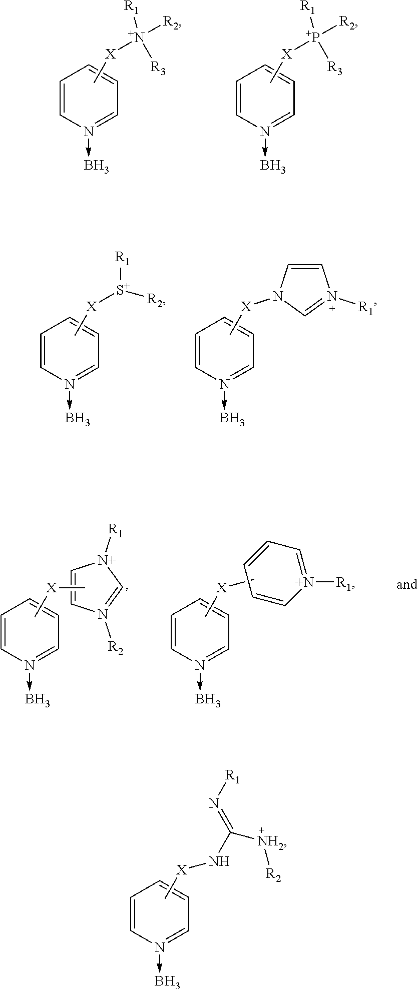
wherein X comprises a generic linker group and R₁, R₂, and R₃ each comprise a generic substituent.

33. (canceled)

34. The composition of claim 31, wherein the cationic moiety comprises any one or more of nitrogen, ammonium, quaternary ammonium, phosphonium, sulfonium, imidazolium, pyridinium, and guanidinium.

35. The composition of claim 31, wherein the substituted pyridine borane complex comprises any one or more of the following structures:

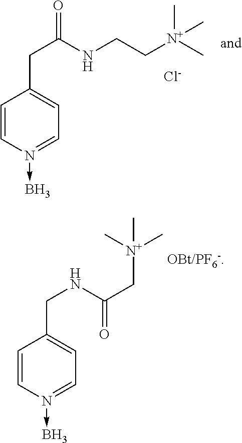

36. A method of using the composition of claim 1, the method comprising:
using a ten-eleven translocation (TET) dioxygenase to oxidize any 5-methylcytosine or 5-hydroxymethylcytosine in the polynucleotide to 5-carboxycytosine or 5-formylcytosine;
using the composition to reduce the 5-carboxycytosine or 5-formycytosine to 5,6-dihydrouracil; and
detecting the 5-methylcytosine or 5-hydroxymethylcytosine using the 5,6-dihydrouracil.


SEQUENCE LISTING (13 sequences)

SEQ ID NO: 1 - DNA, 12 bp, synthetic construct
  source 1..12: mol_type = other DNA
  modified_base 5: mod_base = OTHER; note = hydroxymethylcytosine
  modified_base 12: mod_base = OTHER; note = methylcytosine
  misc_feature 1..12: note = polynucleotide
ccgtnggaccgn

SEQ ID NO: 2 - DNA, 12 bp, synthetic construct
  source 1..12: mol_type = other DNA
  misc_feature 1..12: note = polynucleotide
  modified_base 5: mod_base = OTHER; note = carboxycytosine
  modified_base 12: mod_base = OTHER; note = carboxycytosine
ccgtnggaccgn

SEQ ID NO: 3 - DNA, 12 bp, synthetic construct
  source 1..12: mol_type = other DNA
  misc_feature 1..12: note = polynucleotide
  modified_base 5: mod_base = OTHER; note = formylcytosine
  modified_base 12: mod_base = OTHER; note = formylcytosine
ccgtcggaccgc

SEQ ID NO: 4 - DNA, 12 bp, synthetic construct
  source 1..12: mol_type = other DNA
  misc_feature 1..12: note = polynucleotide
  modified_base 5: mod_base = OTHER; note = formylcytosine
  modified_base 12: mod_base = OTHER; note = carboxycytosine
ccgtnggaccgn

SEQ ID NO: 5 - DNA, 12 bp, synthetic construct
  source 1..12: mol_type = other DNA
  misc_feature 1..12: note = polynucleotide
  modified_base 5: mod_base = OTHER; note = carboxycytosine
  modified_base 12: mod_base = OTHER; note = formylcytosine
ccgtnggaccgn

SEQ ID NO: 6 - DNA, 12 bp, synthetic construct
  source 1..12: mol_type = other DNA
  misc_feature 1..12: note = polynucleotide
  modified_base 5: mod_base = OTHER; note = 5,6-dihydrouracil
  modified_base 12: mod_base = OTHER; note = 5,6-dihydrouracil
ccgtnggaccgn

SEQ ID NO: 7 - DNA, 12 bp, synthetic construct
  source 1..12: mol_type = other DNA
  misc_feature 1..12: note = polynucleotide
ccgttggaccgt

SEQ ID NO: 8 - DNA, 12 bp, synthetic construct
  source 1..12: mol_type = other DNA
  misc_feature 1..12: note = polynucleotide
ggcaacctggca

SEQ ID NO: 9 - DNA, 12 bp, synthetic construct
  source 1..12: mol_type = other DNA
  misc_feature 1..12: note = polynucleotide
ccgtcggaccgc

SEQ ID NO: 10 - DNA, 12 bp, synthetic construct
  source 1..12: mol_type = other DNA
  misc_feature 1..12: note = polynucleotide
ggcagcctggcg

SEQ ID NO: 11 - DNA, 12 bp, synthetic construct
  source 1..12: mol_type = other DNA
  misc_feature 1..12: note = polynucleotide
  modified_base 5: mod_base = OTHER; note = carboxycytosine
  modified_base 10: mod_base = OTHER; note = formylcytosine
ccgtnggacngc

SEQ ID NO: 12 - DNA, 12 bp, synthetic construct
  source 1..12: mol_type = other DNA
  misc_feature 1..12: note = polynucleotide
  modified_base 5: mod_base = OTHER; note = 5,6-dihydrocytidine
  modified_base 10: mod_base = OTHER; note = 5,6-dihydrocytidine
ccgtnggacngc

SEQ ID NO: 13 - DNA, 12 bp, synthetic construct
  source 1..12: mol_type = other DNA
  misc_feature 1..12: note = polynucleotide
  modified_base 5: mod_base = OTHER; note = 5,6-dihydrouracil
  modified_base 10: mod_base = OTHER; note = 5,6-dihydrouracil
ccgtnggacngc